(19) United States
(12) Patent Application Publication
(10) Pub. No.: US 2014/0271694 A1
(43) Pub. Date: Sep. 18, 2014

(54) ANTIGEN-SPECIFIC METHODS OF DIAGNOSING AND/OR TREATING MYOCARDITIS OR INFLAMMATORY CARDIOMYOPATHY

(71) Applicant: Joslin Diabetes Center, Inc., Boston, MA (US)

(72) Inventor: Myra A. Lipes, Brookline, MA (US)

(21) Appl. No.: 14/206,430

(22) Filed: Mar. 12, 2014

Related U.S. Application Data
(60) Provisional application No. 61/779,810, filed on Mar. 13, 2013.

(51) Int. Cl. C07K 14/47

(52) U.S. Cl. CPC C07K 14/47
USPC 424/185.1; 530/350

(57) ABSTRACT

Provided herein are, for example, methods of diagnosing and/or treating inflammatory cardiomyopathy and/or post-myocardial injury autoimmunity. Such therapies involve suppressing an immune response to the alpha isoform of myosin heavy chain in the subject. In some embodiments, this suppression is done selectively.

### GOVERNMENT INTEREST

This invention was made with government support under RO1 HL077554 and RO1 DK072090, awarded by the National Institute of Health. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled SeqListMYRA005PR.txt, created on Mar. 13, 2013, which is 16,204 bytes in size. The information in the electronic format of the Sequence Listing is incorporated herein by reference in its entirety.

### TECHNICAL FIELD

Embodiments provided herein relate to methods of diagnosing and/or treating myocarditis or inflammatory cardiomyopathy, including, for example, post-myocardial injury-induced autoimmunity in subjects.

### BACKGROUND

Myocarditis is an acute or chronic inflammatory disease of heart muscle and a major cause of heart failure due to progression to dilated cardiomyopathy (weakening of the heart muscle). Myocarditis can be produced by a wide variety of injuries to the myocardium, including toxins and drugs (for example, cocaine, interleukin 2) or infectious agents, most commonly including viral (for example, coxsackievirus, adenovirus, HIV, hepatitis C virus), bacterial (for example, diphtheria, meningococcus, psittacosis, streptococcus), rickettsial (for example, typhus, Rocky Mountain spotted fever), fungal (for example, aspergillosis, candidiasis), and parasitic (Chagas disease, toxoplasmosis), as well as giant cell myocarditis, and hypersensitivity reactions to drugs such as antibiotics, sulfonamides, anticonvulsants, and anti-inflammatories (Maron, Circulation, 2006). In addition, it has recently been reported that in autoimmune-prone mice and patients with type 1 diabetes, chronic myocarditis can be the result of “sterile” cardiac injury (not due to infection or toxin, as above) in particular including myocardial infarction (MI). (Gottumukkala, Science Translational Medicine, 2012).

Previous studies on inflammatory heart disease have focused on systemic immunosuppression or humoral immunity with minimal or no therapeutic benefit (Lappe, Journal of Cardiac Failure, 2008), and immunosuppression is generally contraindicated in infectious disease settings. Numerous clinical studies have been performed to non-specifically suppress inflammatory responses to optimize cardiac repair after heart attack (myocardial infarction or “MI”) in patients from the general population. However, the negative results from studies using general immunosuppressants such as methylprednisolone and more recently anti-CD18 (integrin) have underscored the risks of using systemic immunosuppression to interfere with the post-MI inflammatory pathways, which are now understood to play an essential protective role in mediating post-MI healing and cardiac repair (Swirski & Nahrendorf, Science, 2013).

### SUMMARY

In some embodiments, a method is provided for treating post-myocardial injury autoimmunity in a subject. The method can comprise identifying an autoimmune-prone subject who has suffered a myocardial injury, and suppressing an immune response to the alpha isoform of myosin heavy chain (“alpha-myosin”) in the subject. In some embodiments, the method further comprises testing the subject for autoantibodies and/or T-cell responses against alpha myosin. In some embodiments, the autoimmune-prone subject has a disorder selected from the group consisting of at least one of: type I diabetes, celiac disease, thyroiditis, and autoimmune thyroid disease. In some embodiments, the myocardial injury comprises a myocardial infarction. In some embodiments, the autoimmune-prone subject has ischemic heart disease. In some embodiments, the autoimmune-prone subject has received a heart transplant. In some embodiments, the autoimmune-prone subject has inflammatory cardiomyopathy. In some embodiments, the autoimmune-prone subject has been infected or is at risk of being infected with a cardiotropic pathogen. In some embodiments, suppressing the immune response to alpha myosin is not an immunosuppressive therapy. In some embodiments, suppressing the immune response to alpha myosin is achieved by antigen-specific tolerogenic therapy. In some embodiments, suppressing the immune response to alpha myosin is achieved by HLA class II-specific immunointervention. In some embodiments, suppressing the immune response to alpha myosin is achieved by administering an alpha myosin-specific peptide to the subject. In some embodiments, the alpha myosin-specific peptide comprises at least one amino acid unique to the alpha isoform (SEQ ID NO: 1) and not found in the beta isoform. In some embodiments, the alpha myosin-specific peptide sequence is contained within a S2 or a LMM domain of alpha myosin. In some embodiments, the alpha myosin-specific peptide comprises a contiguous stretch of at least 8 amino acids of SEQ ID NO: 1 and wherein the alpha myosin-specific peptide includes one or more of the following amino acids: 2, 4, 5, 8, 14, 33, 34, 35, 36, 37, 44, 52, 60, 65, 77, 103, 107, 110, 111, 135, 136, 197, 205, 209, 210, 211, 212, 213, 283, 304, 314, 319, 320, 327, 335, 348, 349, 350, 360, 367, 381, 417, 422, 424, 425, 435, 554, 562, 574, 586, 592, 596, 608, 618, 619, 623, 624, 627, 630, 631, 632, 633, 634, 636, 639, 640, 666, 680, 682, 730, 744, 790, 794, 797, 799, 801, 803, 806, 807, 811, 847, 853, 859, 861, 864, 901, 954, 1014, 1021, 1023, 1025, 1076, 1079, 1081, 1086, 1088, 1089, 1091, 1092, 1094, 1095, 1101, 1103, 1104, 1113, 1250, 1251, 1258, 1261, 1263, 1265, 1268, 1272, 1275, 1277, 1290, 1294, 1309, 1314, 1325, 1401, 1520, 1521, 1524, 1525, 1537, 1540, 1593, 1613, 1733, 1734, 1737, 1741, 1826, 1860, 1931, 1933, 1934, 1935, 1936, 1937, 1938, and 1939 of SEQ ID NO: 1. In some embodiments, suppressing the immune response to alpha myosin is achieved by administering a mimotope that mimics an epitope that is unique to the alpha isoform of alpha myosin (SEQ ID NO: 1) and not found in the beta isoform. In some embodiments, suppressing the immune response to alpha myosin comprises vaccinating the subject against a peptide unique to alpha myosin. In some embodiments, suppressing the immune response to alpha myosin is achieved by inducing alpha myosin-specific T regulatory cells. In some embodiments, suppressing the immune response to alpha myosin is achieved by a small molecule inhibitor to a HLA class II peptide binding pocket. In some embodiments, the small molecule inhibitor comprises tetraazatricyclododecane (TATD). In some embodiments, the subject is a human.

In some embodiments, a method for treating post-myocardial injury autoimmunity is provided. The method can comprise identifying a subject who has been infected or is at risk of being infected with a cardiotropic pathogen and suppressing an immune response to the alpha isoform of myosin heavy chain (“alpha-myosin”) in the subject. In some embodiments, suppressing the immune response to alpha myosin is not an immunosuppressive therapy. In some embodiments, the method further comprises testing the subject for autoantibodies against alpha myosin, T-cell responses against alpha myosin, or both. In some embodiments, suppressing the immune response to alpha myosin is achieved by antigen-specific tolerogenic immunointervention. In some embodiments, suppressing the immune response to alpha myosin is achieved by HLA class II-specific immunointervention. In some embodiments, the subject has evidence of inflammatory cardiomyopathy due to at least one of Chagas' cardiomyopathy, post-viral myocarditis, or other cardiotrophic pathogens. In some embodiments, suppressing the immune response to alpha myosin is achieved by administering an alpha myosin-specific peptide to the subject. In some embodiments, the alpha myosin-specific peptide comprises at least one amino acid unique to the alpha isoform (SEQ ID NO: 1) and not found in the beta isoform. In some embodiments, the alpha myosin-specific peptide sequence is contained within a S2 or a LMM domain of alpha myosin. In some embodiments, the alpha myosin-specific peptide comprises a contiguous stretch of at least 8 amino acids of SEQ ID NO: 1, and wherein the alpha myosin-specific peptide includes one or more of the following amino acids: 2, 4, 5, 8, 14, 33, 34, 35, 36, 37, 44, 52, 60, 65, 77, 103, 107, 110, 111, 135, 136, 197, 205, 209, 210, 211, 212, 213, 283, 304, 314, 319, 320, 327, 335, 348, 349, 350, 360, 367, 381, 417, 422, 424, 425, 435, 554, 562, 574, 586, 592, 596, 608, 618, 619, 623, 624, 627, 630, 631, 632, 633, 634, 636, 639, 640, 666, 680, 682, 730, 744, 790, 794, 797, 799, 801, 803, 806, 807, 811, 847, 853, 859, 861, 864, 901, 954, 1014, 1021, 1023, 1025, 1076, 1079, 1081, 1086, 1088, 1089, 1091, 1092, 1094, 1095, 1101, 1103, 1104, 1113, 1250, 1251, 1258, 1261, 1263, 1265, 1268, 1272, 1275, 1277, 1290, 1294, 1309, 1314, 1325, 1401, 1520, 1521, 1524, 1525, 1537, 1540, 1593, 1613, 1733, 1734, 1737, 1741, 1826, 1860, 1931, 1933, 1934, 1935, 1936, 1937, 1938, and 1939 of SEQ ID NO: 1. In some embodiments, suppressing the immune response to alpha myosin is achieved by administering a mimotope that mimics an epitope that is unique to the alpha isoform of alpha myosin (SEQ ID NO: 1) and not found in the beta isoform. In some embodiments, suppressing the immune response to alpha myosin comprises vaccinating the subject against a peptide(s) unique to alpha myosin. In some embodiments, suppressing the immune response to alpha myosin is achieved by inducing alpha myosin-specific T regulatory cells. In some embodiments, suppressing the immune response to alpha myosin is achieved by a small molecule inhibitor to a HLA class II peptide binding pocket. In some embodiments, the small molecule inhibitor comprises tetraazatricyclododecane (TATD). In some embodiments, the subject is a human.

In some embodiments, a method of vaccinating subjects at risk for developing Chagas' cardiomyopathy is provided. The method can comprise identifying a subject at risk of developing Chagas' cardiomyopathy, and administering to the subject a compound to suppress an immune response to the alpha isoform of myosin heavy chain (“alpha-myosin”) in the subject. In some embodiments, the compound comprises a vaccine comprising a peptide(s) unique to alpha myosin in comparison to beta myosin. In some embodiments, identifying a subject at risk comprises identifying a subject who is, or is going to be, exposed to an environment in which Chagas disease is endemic.

In some embodiments, a method of heart transplantation is provided. The methods can comprise transplanting a heart to a subject and suppressing an immune response to an alpha isoform of myosin heavy chain (“alpha-myosin”) in the subject.

In some embodiments, a method of treating post-myocardial injury autoimmunity is provided. The method can comprise identifying a subject who has received or will receive a heart transplantation, and suppressing an immune response to the alpha isoform of myosin heavy chain (“alpha-myosin”) in the subject.

In some embodiments, an alpha myosin-specific peptide is provided. The peptide comprises a contiguous stretch of at least 8 amino acids of SEQ ID NO: 1 and wherein the alpha myosin-specific peptide includes one or more of the following amino acids: 2, 4, 5, 8, 14, 33, 34, 35, 36, 37, 44, 52, 60, 65, 77, 103, 107, 110, 111, 135, 136, 197, 205, 209, 210, 211, 212, 213, 283, 304, 314, 319, 320, 327, 335, 348, 349, 350, 360, 367, 381, 417, 422, 424, 425, 435, 554, 562, 574, 586, 592, 596, 608, 618, 619, 623, 624, 627, 630, 631, 632, 633, 634, 636, 639, 640, 666, 680, 682, 730, 744, 790, 794, 797, 799, 801, 803, 806, 807, 811, 847, 853, 859, 861, 864, 901, 954, 1014, 1021, 1023, 1025, 1076, 1079, 1081, 1086, 1088, 1089, 1091, 1092, 1094, 1095, 1101, 1103, 1104, 1113, 1250, 1251, 1258, 1261, 1263, 1265, 1268, 1272, 1275, 1277, 1290, 1294, 1309, 1314, 1325, 1401, 1520, 1521, 1524, 1525, 1537, 1540, 1593, 1613, 1733, 1734, 1737, 1741, 1826, 1860, 1931, 1933, 1934, 1935, 1936, 1937, 1938, and 1939 of SEQ ID NO: 1. In some embodiments, the peptide comprises a T cell immunodominant epitope.

### BRIEF DESCRIPTION OF DRAWINGS

FIG. 1 is a comparison of the human alpha- and beta-myosin heavy chain (MyHC) amino acid sequence (Accession numbers P13533 and P12883). The complete alpha-myosin HC (“MYH6”) sequence is shown on the top line (SE and non-identical beta myosin HC (“MYH7”) sequence amino acids are shown on the bottom line. Sequences of at least 10 contiguous amino acids that contain at least one amino acid unique to alpha myosin HC have been underlined in the alpha myosin HC amino acid sequence.

FIG. 2A is a set of serial sections showing infarct expansion and lymphocytic infiltrates in the peri-infarct zone only in non-obese diabetic (NOD) mice (left panel, boxes). Immunohistochemical (IHC) staining showing that the cardiac infiltrates (middle panel) consist of B220 B cells, CD4 and CD8 T cells, similar in composition to “insulitis” lesions in the pancreas (right panel).

FIG. 2B is a set of images showing extension of the infiltrates into the non-infarcted myocardium and poor infarct healing in a NOD mouse heart 8 wk post-MI (upper panel).

FIG. 3A is a set of images showing indirect immunofluorescence staining on normal mouse heart sections using sera from a post-MI NOD mouse, a humanized DQ8+NOD mouse with myocarditis, and a post-MI control C57BLU6 (B6) mouse, as indicated. Green indicates serum staining; blue indicates nuclear staining (DAPI).

FIG. 3B are immunoblots containing normal heart extracts probed with sera from NOD and control B6 mice pre-MI and 3 wk post-MI, using a β-actin mAb control, showing that circulating autoantibodies from post-MI NOD mice not only recognized cardiac myosin heavy chain (MyHC) but also a second ˜100 kDa protein detectable only in detergent (SDS)-containing lysates.

FIG. 3C is a Western blot that was probed with post-MI NOD serum showing that the −100 kDa protein was expressed at high levels in cardiac muscle and skeletal muscle but was absent in lung, liver, kidney and brain. (He: heart; Sk: skeletal muscle; Lu: lung; Li: liver; Ki: kidney; Sp: spleen; Br: brain).

FIG. 3D shows the sequenced peptides for the −100 kDa obtained from mass spectrometry, which demonstrated 100% identity to mouse Actn2 (AAF76325.1).

FIG. 3E is an analysis showing the expression and purification of recombinant mouse Actn2 which was done using a polyhistidine-tagged fusion protein in Escherichia coli (left panel); and a Western blot confirming that sera from post-MI NOD mice recognized recombinant mouse Actn2 identically to the native Actn2 contained in heart lysates (He) (right panel).

FIG. 4A is a set of immunoblots containing normal heart extracts probed with sera from NOD and control B6 mice 8 wk post-MI (n=6, respectively) and for comparison, sera from a single NOD MI mouse 4 wk post-MI, showing that the autoantibodies from post-MI NOD mice (but not non-diabetic B6 mice) are reactive to myosin heavy chain (MyHC; arrow) and Actn2.

FIG. 4B is a set of immunoblots probed with sera from 12 wk post-MI control B6 mice (n=8 mice per group), showing that cardiac autoantibodies were absent or barely detectable in the post-infarcted control B6 mice.

FIG. 4C is a dot plot showing titers and prevalence of cardiac myosin (CM) autoantibodies measured by ELISA in NOD post-MI mice (n=17), NOD microinfarcted mice (n=15), Sham-MI [NOD mice that received just open-chest thoracotomy, but no LAD coronary occlusion, (n=10)], and control B6 post-MI mice (n=10).

FIG. 5 is a set of images showing histological heart sections from microinfarcted NOD and B6 mice obtained 4 wk post-microinfarction stained with H&E and Masson's trichrome, showing distinct regions of fibrosis stained in blue.

FIG. 6 is a set of images showing histological heart sections from sham-MI NOD and B6 mice 4 wk post-sham-MI, and a sham-MI NOD mice 8 wk post-sham-MI, that have been stained with H&E and Masson's trichrome, showing the absence of fibrosis in sham-MI mice.

FIG. 7A is a dot plot showing that post-MI NOD mice developed strong Th1 effector responses to cardiac myosin (CM), similar in magnitude to those found in humanized DQ8+NOD mice with myocarditis. Splenocytes from 3 wk post-MI NOD and B6 mice, and from unmanipulated NOD mice and DQ8+NOD mice with spontaneous myocarditis (‘Myocarditis’), were stimulated in vitro with 25 μg/ml of CM, and IFN-γ-producing T cells were detected by ELISPOT assays. *P<0.05: **P<0.001.

FIG. 7B is a set of bar graphs showing dose-dependent T cell responses to CM in cardiac-draining lymph nodes (LN) of NOD mice 8 wk post-MI. T cell responses in cardiac-draining LN and mesenteric LNs from NOD and B6 mice were compared. Cardiac-draining LN (upper panel) and mesenteric LN (lower panel) cells from 7 NOD and 6 B6 (pooled) mice 8 wk post-MI were stimulated with the indicated amounts of CM and T cell responses were detected by ³[H]thymidine incorporation.

FIG. 7C is a set of dot plots showing the development of anti-CM and anti-Actn2 immunoglobulin (Ig) isotype switching, with similar cardiac myosin-specific autoantibody isotype switching profiles in post-MI NOD mice and DQ8+NOD mice with spontaneous myocarditis. Serial sera from post-MI NOD and B6 mice (n=5 mice/group) were obtained on a biweekly basis (w0=pre-MI bleed; w=wk post-MI) and serial sera from the same humanized DQ8⁺NOD mice (‘Myocarditis’) (n=5) taken between ages 4-6 wk and again at 7-12 wk, were used for a comparison.

FIGS. 8A-B is an analysis of T-cell responses to Actn2 showing that no increased T cell responses to Actn2 were observed in post-MI NOD mice. Splenocytes from 4 wk post-MI NOD mice were obtained and T cell responses to Actn2 were measured by IFN-γ ELISPOT (FIG. 8A) and proliferation (FIG. 8B) assays. Unmanipulated NOD wild type (WT) mice were used as controls and mouse cardiac myosin (CM) was used as a positive control antigen for Actn2 ELISPOT assays. **P=0.0032.

FIG. 9 is a set of dot plots showing that cardiac myosin-specific IgG3 and IgA responses were undetectable in post-MI NOD and B6 mice. As shown in FIG. 7C, serial sera from post-MI NOD and B6 mice (n=5 mice/group) were obtained on a biweekly basis (w0=pre-MI bleed; w=wk post-MI) and serial sera from the same DQ8⁺NOD mice (‘Myocarditis’) (n=5) taken between ages 4-6 wk and again at 7-12 wk, were used for a comparison.

FIG. 10A is a table listing the features of the pre- and post-MI (TOM+TA− or TOM−TA+) DQ8+NOD and TOM+TA+DQ8+NOD mice. The findings in this table and FIGS. 10B and 10C demonstrate that induction of immune tolerance to α-myosin HC prevents the development of post-MI autoimmunity.

FIG. 10B is set of immunoblots containing normal mouse heart extracts probed with sera from the indicated mice, showing markedly reduced or absent cardiac autoantibody production in TOM+TA+DQ8+NOD mice that were made tolerant to α-MyHC. For NOD WT control, Pre-MI=pre-bleed; Post-MI=21 d post-MI; For (TOM+TA+ or TOM+TA− or TOM−TA+) DQ8+NOD mice sera, 1=pre-MI bleed; 2=20 d post-MI bleed. Cardiac antigens that were recognized by the sera samples are listed on the right, where MyHC* is a MyHC degradation product. The open arrow indicates Actn2, which was detected in post-MI NOD sera, but not in (TOM+TA− or TOM−TA+) DQ8+NOD mice.

FIG. 10C are histological heart sections from a 20 d post-MI control TOM−TA+DQ8+NOD mouse (31282) and the thymic α-MyHC-expressing TOM+TA+DQ8+NOD littermate (31286) that have been stained with H&E, showing significant pathology in the control TOM−TA+DQ8+NOD mouse (31282) (right panel) and the absence of lymphocytric infiltration and instead normal dense scar formation in an α-MyHC-tolerant TOM+TA+DQ8+NOD littermate (31286) (left panel).

FIG. 11 is a Western blot analysis of cardiac myosin autoreactivity in humans, comparing autoreactivity in 3 consecutively healthy control subjects (“Healthy controls”) to myocarditis patient M-1 (“Myocarditis”) and post-MI T1D patient T1D-16 (“T1D+MI+”). Sera and peroxidase conjugated secondary antibody were used at 1:1,000 and 1:10,000 dilutions, respectively. Lanes 1-3 contained, respectively, 2.5 ug of human left ventricle myofibrillar extract (lane 1); human skeletal muscle myofibrillar extract (lane 2); and human ventricular SDS lysate (lane 3). Lanes 4-5 contained, respectively, 0.25 ug of purified human ventricle cardiac myosin (lane 4) and purified human cardiac troponin I/cardiac troponin T (lane 5).

FIG. 12A is a schematic showing constructs used in cardiac myosin autoantibody radioimmunoprecipitation assays. FL, full length; fragments corresponding to the S1, S2 and LMM domains of human α-myosin HC.

FIG. 12B is a set of dot plots showing the prevalence of autoantibodies to the S1 domain of human α-MyHC (α-MyHC S1), human α-Actinin-2 (Actn2), and human cardiac troponin I (cTnI) in healthy control (HC), post-MI T1D (T1D+), myocarditis (Myo), and post-MI type 2 diabetic (T2D+MI+) subjects. The dashed lines indicate the upper limit of the normal range, defined as the mean of the antibody index values obtained from the healthy controls plus 3 SD.

FIG. 13A is a set of cardiac magnetic resonance images (MRI) confirming the presence of myocarditis in a post-MI T1D patient who tested positive for cardiac autoantibodies against alpha-myosin HC. A cardiac MRI from a cardiac autoantibody-positive patient with post-viral myocarditis is shown as a positive control. Cardiac MRI of 61-yr-old post-MI T1D patient (T1D-8) with progressive heart failure and dilated cardiomyopathy [left ventricular ejection fraction (LVEF)=18%] is shown on the left panel and positive control patient (M-5) with acute myocarditis (LVEF=42%, normal LV size) is shown on the right panel.

FIG. 13B is a set of cardiac MRIs showing myocardial inflammation in T1D patient (T1D-8) by comparing T2* images before (FIGS. 13B (section i) and 13B (section ii), at echo times of 1.4 and 21 msec, respectively) and after (FIGS. 13B (section iii) and 13B (section iv), at echo times of 1.4 and 21 msec, respectively) injection of the superparamagnetic iron oxide-based contrast agent, Ferumoxytol.

FIG. 13C is a set of decay curves showing evidence of accumulation of iron oxide in the myocardium 24 hours post injection of Ferumoxytol, as indicated by the T2* decay constants. The upper panel shows the decay curve 24 msec from before injection and the lower panel shows the decay curve 9.4 msec 24 hours after injection.

FIGS. 14A-D are data showing that Clone E, the pathogenic DQ8-restricted CD4⁺ T cell clone isolated from a myocarditic heart (Lv H et al., J Clin Invest, 2011), is a highly specific pathogenic T cell clone restricted to DQ8 and that it recognizes human cardiac myosin. FIG. 14A shows T cell responsiveness to cardiac myosin (CM) and soleus myosin (SM) as measured by ³[H]thymidine incorporation. FIG. 14B shows a schematic of the percent amino acid sequence identity between mouse and human alpha myosin HC and their predominant site of expression. FIG. 14C is a bar graph showing that Clone E responds to cardiac myosin only in the context of DQ8+ APCs and not NOD WT (I-Ag7) APCs. FIG. 14D is a graph showing that Clone E recognizes the alpha isoform of human cardiac myosin (huCM-A) in proliferation assays (SI=stimulation index).

FIGS. 15A-C are data showing that the small molecule, tetraazatricyclododecane (TATD), that was predicted to bind pocket 6 of the DQ8 peptide-binding groove (Michels et al, Journal of immunology, 2011), inhibits cardiac myosin presentation by DQ8 to T cells. FIGS. 15A and B are graphs showing that T cell response to insulin B9:23 peptide, measured by IL-2 concentration, is blocked by TATD in a dose dependent manner, for DQ8 and I-Ag7 (Michels et al., Journal of Immunology, 2011). FIG. 15C is a graph showing that T cell response to cardiac myosin stimulation is blocked in vitro by TATD is a dose-dependent manner, in a manner similar to the blocking effect on insulin B9:23 presentation to DQ8. In this assay, T cell responses were measured using our cardiac myosin-specific DQ8-restricted (Clone E) T cell hybridoma that was engineered to produce β-galactosidase following T-cell receptor-antigen stimulation.

FIGS. 16A-B are schematics and data showing the creation of a clone E hybridoma with LacZ and GFP as readouts of Ag-specific T cell stimulation. FIG. 16A is a schematic illustrating the creation of a lacZ and GFP-inducible α-Myosin HC-specific CD4⁺ T-cell hybridoma made by fusing Clone E with a BWZ.36 hybridoma partner with NFAT-driven expression of Green Flourescence Protein (GFP) and LacZ (Karttunen J et al., Proc Natl Acad Sci USA), to generate the hybridoma, BWZ.36.CE.GFP. FIG. 16B is a flow cytometry analysis showing that the BWZ.36.CE.GFP hybridoma responds to cardiac myosin but not the no antigen negative control.

FIGS. 17A-C are schematics and results showing that the DQ8-restricted disease-causing epitope of alpha myosin HC resides in the S2/LMM domain. FIG. 17A is a schematic showing the S1, S2 and LMM regions of α-myosin HC. FIG. 17B shows the creation and purification of subfragments S1 and S2/LMM, using enzymatic digestion and ion-exchange chromatography (CM=cardiac myosin control). FIG. 17C is a flow cytometry analysis showing that Clone E-GFP hybridoma responds to the cardiac myosin and S2/LMM subfragments, but not the S1 subfragment. However, the fragment of “S2-LMM” used herein contains a small region of peptides within the junction of S1/S2 that contains some amino acids unique to alpha myosin-HC and which belong to S1.

FIG. 18 is a diagram showing the proposed pathogenesis of post-infarction autoimmunity (PIA) in a subject with type 1 diabetes. [1] shows the escape of high-avidity (‘forbidden’) cardiac myosin (CM)-specific naïve CD4+ T cells from the thymus due to the thymus lacking alpha-myosin HC expression; [2] shows the necrotic release of cardiac myosin following a myocardial infarction; [3] shows the innate inflammation following a myocardial infarction, characterized by production of proinflammatory cytokines and migration of macrophages and dendritic cells (DCs) into the infarct zone and resultant activation of DCs; [4] shows the defective immunoregulation in an autoimmune prone host, such as NOD mice or type 1 diabetes patients, wherein cardiac myosin-specific CD4+ T cells become persistently activated, migrate out of the cardiac lymph node, and migrate back into the heart, causing additional injury; [5] shows the vicious cycle in which the additional injury causes additional activation and expansion of CM-specific CD4+ T cells, leading to chronic myocarditis with impaired infarct healing and heart failure (Lv and Lipes, Trends in Cardiovascular Medicine, 2012).

### DETAILED DESCRIPTION

Described herein are methods for treating inflammatory cardiomyopathy (myocarditis) or post-myocardial injury autoimmunity by HC and suppressing an immune response to alpha myosin HC. In some embodiments, the method comprises testing for autoantibodies and/or T-cell responses against alpha myosin HC.

### DEFINITIONS

As used herein, the terms “inflammatory cardiomyopathy” and “myocarditis” are used interchangeably and refer to an acute or chronic inflammatory process affecting the myocardium that can be produced by a wide variety of injuries to the myocardium, including toxins and drugs (for example, cocaine, interleukin 2) or infectious agents, most commonly including viral (for example, coxsackievirus, adenovirus, HIV, hepatitis C virus), bacterial (for example, diphtheria, meningococcus, psittacosis, streptococcus), rickettsial (for example, typhus, Rocky Mountain spotted fever), fungal (for example, aspergillosis, candidiasis), and parasitic (Chagas disease, toxoplasmosis), giant cell myocarditis, and hypersensitivity reactions to drugs such as antibiotics, sulfonamides, anticonvulsants, and anti-inflammatories; as well as “sterile” myocardial injury (not due to infection or toxin, as above) such as myocardial infarction or ischemic heart disease in autoimmune-prone subjects such as patients with type 1 diabetes; as well as myocardial injury related to heart transplantation. Additional symptoms or disorders such as dilated cardiomyopathy or progressive heart failure may also be present, but are not required.

As used herein, the term “post-myocardial injury autoimmunity” refers to myocardial autoimmunity following any of the myocardial injuries described above. The term encompasses autoimmune myocarditis and/or autoimmune inflammatory cardiomyopathy, as well as post-infarction autoimmune syndrome (“PIA syndrome”). The phrase “post-myocardial injury autoimmunity” and “post-cardiac injury autoimmunity” are used interchangeably.

As used herein, the term “post-infarction autoimmune syndrome” (or “PIA syndrome”) refers to autoimmune myocarditis following a myocardial infarction or associated with ischemic heart disease (Gottumukkala, Science Translational Medicine, 2012; Lv and Lipes, Trends in Cardiovascular Medicine, 2012). Additional symptoms or disorders such as poor infarct healing, dilated cardiomyopathy, or progressive heart failure may also be present, but are not required.

As used herein, the term “post-infarction autoimmunity” encompasses myocardial autoimmunity following a myocardial infarction or associated with ischemic heart disease.

As used herein, “treatment” or “treating” and variations thereof refers to any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the methods of the present invention. In addition to traditional forms of treatment, preventative and/or prophylactic treatments are also encompassed within the above term.

The terms “effective amount” and “effective to treat,” as used herein, refer to an amount or a concentration of one or more compounds or a pharmaceutical composition described herein utilized for a period of time (including acute or chronic administration and periodic or continuous administration) that is effective for the recited treatment, for example, for reducing one or more symptoms or clinical signs, and returning the subject to normal or more normal function. In addition, in some embodiments the methods of treatment may result in the induction of immune tolerance to alpha myosin HC.

The term “suppressing” does not require complete inhibition. The term denotes that at least some amount of detectable decrease has occurred. The phrase “suppressing the immune response” encompasses inducing tolerance to alpha myosin HC.

The term “vaccinate” does not require that the subject experience no adverse side effects of aspects of the disorder that the subject is vaccinated against. Rather, the term denotes that one or more of the symptoms of a disease or disorder are ameliorated or otherwise beneficially altered.

The term “subject” is used throughout the specification to describe an animal, human or non-human, to whom treatment according to the methods of the present invention is provided. Veterinary and non-veterinary applications are contemplated. The term includes, but is not limited to, mammals, for example, humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Cardiac Alpha Myosin

Myosin is a large family of motor proteins. It is a hexameric protein containing two heavy chain subunits, and four light chain subunits. Cardiac myosin refers to an isoform of myosin expressed in cardiac muscles. For example, alpha myosin HC (MYH6) is a cardiac-specific isoform. Most previous clinical investigations on inflammatory human heart disease have focused on the beta isoform (MYH7) of myosin since it is the most abundant isoform (−90% of total myosin) in the human heart.

When myosin is exposed to the proteolytic enzyme trypsin, fragmentation occurs to yield heavy meromyosin (HMM) and light meromyosin (LMM). HMM containing the head and a short tail can be further split by proteolytic enzymes, such as papain or chymotrypsin, into subfragment 1 (S I) and subfragment 2 (S2).

Alpha myosin HC polypeptides and antigenic fragments thereof and nucleic acids encoding alpha myosin HC polypeptides and antigenic fragments thereof are useful in the methods described herein. Exemplary alpha myosin HC amino acid sequences can be found at, for example, Genbank Accession Nos. NP_{—}002462.2 (human) and P_{—}034986.1 (mouse). The various domains within alpha myosin HC are known in the art. Exemplary alpha myosin HC nucleic acid sequences can be found at Genbank Accession Nos NM_{—}002471.3 (human) and NM_{—}010856.4 (mouse). The terms “alpha myosin HC,” “α-myosin HC,” “α-MyHC,” and the like are used interchangeably herein.

Alpha myosin HC and immunogenic fragments thereof and nucleic acids encoding alpha myosin HC and fragments thereof can be generated using the methods known in the art or described above. Cardiac myosin can also be purified from cardiac tissues (for example, from human or mouse) using methods known in the art. See, for example, Caforio et al, Circulation, 1992, 85: 1734-1742. As detailed herein, especially useful are peptides of alpha myosin HC that contain amino acid positions that are different from their corresponding position in the beta isoform of myosin. Such peptides can be useful as a therapeutic for the disorders noted herein. Examples of such peptides are discussed in more detail below and some options are shown in FIG. 1.

### Methods of Treatment

Patients with type 1 diabetes (T1D) suffer excessive mortality following an MI (for example ˜13-fold increase in age-adjusted mortality rates compared to the general population). In addition, infection by cardiotropic pathogens, such as Coxsackievirus B3 or Trypanosoma cruzi, is also a significant cause of heart failure. For example, Chagas disease caused by Trypanosoma cruzi is endemic in Latin America, affecting 7.7 million people. The World Health Organization estimates that currently there are 18 million infected cases worldwide. An estimated 30-40% of those people develop Chagas' cardiomyopathy, which is a major cause of death and leading cause of cardiomyopathy worldwide (Liu and Baughman, Braunwald's Heart disease, 9^{th} Edition, 2012).

Provided herein are methods of treating inflammatory cardiomyopathy or post-myocardial injury autoimmunity in a subject.

Generally, the method includes identifying a subject in need (for example, an autoimmune-prone subject who has suffered (or is likely to suffer) a myocardial infarction or a subject who has been infected with a cardiotropic pathogen). In some embodiments, the method can further include testing the subject for autoantibodies and/or T cell responses against alpha myosin HC. If the subject tests positive for this, one can then administer a therapy to the subject to suppress the immune responses to alpha myosin HC. In some embodiments, the therapy is selective for the suppression of the response to alpha myosin HC, rather than a general suppression of the immune response. In some embodiments, the therapy is selective for alpha myosin HC over beta myosin or other possible targets. In some embodiments, any of the methods and/or compositions provided herein directed to suppressing an immune response to alpha myosin HC can be adequately selective for the suppression of the response to alpha myosin HC specifically, and will not result in a general suppression of immune responses. Thus, in some embodiments, the method and/or compositions consist essentially of suppressing the immune responses to alpha myosin HC.

In some embodiments, suppressing the immune response to alpha myosin HC may be done alone or in combination with other treatments. In some embodiments, suppressing the immune response to alpha myosin HC may be done prior to initiation or completion of testing of the subject for autoantibodies and/or T cell responses against alpha myosin HC. In some embodiments, testing will typically be completed and a positive result for alpha myosin HC autoantibodies and/or T-cell responses against alpha myosin HC will be obtained prior to initiating suppression of the immune response to alpha myosin HC. In some embodiments, testing of the subject for autoantibodies and/or T cell responses against alpha myosin HC is not required.

In some embodiments, suppression of the immune response against alpha myosin HC is not an immunosuppressive therapy. Immunosuppressive therapies are known in the art and include the following: administration of rituximab; anti-CD3 antibodies; IFN-alpha; IV immunoglobulin; immunoabsorption therapy; azathioprine; thymomodulin; tacrolimus; sirolimus; mycophenolate; fingolimod; and myriocin. Immunosuppressive therapies for non-diabetic subjects also include immune suppressive glucocorticoinds such as prednisone and cyclosporine. See also Rose and Baughman, “Myocarditis and dilated cardiomyopathy.” In: Rose and Mackay, eds. The Autoimmune Diseases. (Boston, Mass., USA: Academic Press; 2006:875-888). Such therapies can prove problematic for subjects after a MI. In contrast, suppressing a specific immune response to alpha myosin HC is expected to provide the benefits without as many or any of the disadvantages present from a general immunosuppressive therapy.

In some embodiments, suppressing the immune response to alpha myosin HC is achieved by generating antigen-specific tolerance. Antigen-specific tolerogenic immunointervention can provide an effective means of controlling the autoimmune response via induction or restoration of alpha-myosin HC-specific immune tolerance without the carrying risk of systemic immune suppression which would impair the ability to fight infection or tumors. Antigen-specific tolerogenic immunointervention generally encompasses peptide-based therapies, with or without adjuvants, administration of dendritic cell (DC)-targeted compounds, or DNA and peptide-major histocompatibility complex (pMHC)-based vaccines (Clemente-Ceres, Cold Spring Harbor Press, 2012).

In some embodiments, suppressing the immune response is achieved by HLA class II-specific immunointervention. Such therapies can include small molecules predicted to bind to specific structural pockets (p1, p4, p6, and p9) along the peptide binding groove of MHC class II molecules by high-throughput molecular docking (Michels, Journal of Immunology, 2011).

In some embodiments, suppressing an immune response against alpha myosin HC is achieved by administering an alpha myosin HC-specific peptide(s) to the subject. Any peptide or combination of peptides that is specific to alpha myosin HC can be utilized. In some embodiments, the peptides or combination of peptides administered need not be unique to alpha myosin HC.

In some embodiments, the alpha myosin HC-specific peptide(s) comprises amino acids unique to the alpha isoform (i.e. that are not found in the beta isoform). FIG. 1 shows a comparison of the amino acid sequences of alpha myosin HC and beta myosin HC. Only 143 of the 1939 amino acids in alpha myosin HC are unique to the alpha isoform. In some embodiments, peptide sequences that are at least 8 amino acids long and contain at least one amino acid unique to alpha myosin HC can be used. In addition, alpha myosin HC “mimetope” peptides (a peptide which mimics the structure of an epitope) can be used (von Boehmer and Daniel, Nature Reviews Drug Discovery, 2013). FIG. 1 illustrates exemplary peptide sequences that fit these criteria. The underlined peptide sections in FIG. 1 show some embodiments of peptide sequences that are at least 11 amino acids in length, and encompass at least one difference between alpha and beta myosin HC. The displayed embodiments also include longer peptides where there are clusters of such differences close to one another. The underlined sequences are only representative peptides and/or epitope sections, and do not limit the disclosure provided herein. The peptide will include at least one epitope. In some embodiments, any length of peptide containing at least one amino acid unique to alpha myosin HC may be used in accordance with this embodiment. In some embodiments, the peptides can be 5, 6, 7, 8, 9, 10, 11, 12, 13, 14, 15, 16, 17, 18, 19, 20 or more amino acids in length. In some embodiments, T-cell epitopes presented by MHC class II molecules can be 13-17 amino acids in length. In some embodiments, the peptides are recognized by CD4+ T cells.

In some embodiments, an alpha myosin-specific peptide is provided. The peptide comprises a contiguous stretch of amino acids of SEQ ID NO: 1 and wherein the alpha myosin-specific peptide includes one or more of the following amino acids: 2, 4, 5, 8, 14, 33, 34, 35, 36, 37, 44, 52, 60, 65, 77, 103, 107, 110, 111, 135, 136, 197, 205, 209, 210, 211, 212, 213, 283, 304, 314, 319, 320, 327, 335, 348, 349, 350, 360, 367, 381, 417, 422, 424, 425, 435, 554, 562, 574, 586, 592, 596, 608, 618, 619, 623, 624, 627, 630, 631, 632, 633, 634, 636, 639, 640, 666, 680, 682, 730, 744, 790, 794, 797, 799, 801, 803, 806, 807, 811, 847, 853, 859, 861, 864, 901, 954, 1014, 1021, 1023, 1025, 1076, 1079, 1081, 1086, 1088, 1089, 1091, 1092, 1094, 1095, 1101, 1103, 1104, 1113, 1250, 1251, 1258, 1261, 1263, 1265, 1268, 1272, 1275, 1277, 1290, 1294, 1309, 1314, 1325, 1401, 1520, 1521, 1524, 1525, 1537, 1540, 1593, 1613, 1733, 1734, 1737, 1741, 1826, 1860, 1931, 1933, 1934, 1935, 1936, 1937, 1938, and 1939 of SEQ ID NO: 1. In some embodiments, the peptide comprises a T cell immunodominant epitope.

In some embodiments, the unique amino acid is towards the middle of the peptide sequence. In some embodiments, the unique amino acid is towards the N-terminus of the peptide sequence. In some embodiments, the unique amino acid is towards the C-terminus of the peptide sequence. In some embodiments, the unique amino acid is at the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, or 20^{th} position of the peptide. In some embodiments, more than one unique amino acid is present in the peptide, for example, 1, 2, 3, 4, 5, 6, 7, or 8 amino acids that are different in the alpha and beta myosin sequences can be present within one peptide. In some embodiments, the peptide comprises one or more of the above arrangements. In some embodiments, one or more of the above peptides is no longer than 20 amino acids in length. In some embodiments, the peptide includes at least one of the unique regions in FIG. 1, and comprises an immunodominant T cell epitope.

In some embodiments, since the epitope or epitopes targeted by CD4+ T cells in a given individual can be influenced by their HLA class II genotype (DR-DQ), a cocktail of peptides predicted to bind to that patient's HLA class II molecules can also be personalized based on the person's HLA genotype. In some embodiments, the peptides could be strongly agonistic mimetopes of the natural alpha myosin HC epitope.

In some embodiments, any method of administering the alpha myosin HC-specific peptide(s) or peptide(s) may be used, for example, administration may be done by intramuscular injection, by nasal administration, by oral administration, or the like.

In some embodiments, the dosage of the peptide is an amount effective to treat and/or prevent inflammatory cardiomyopathy, post-myocardial injury autoimmunity, and/or post-infarction autoimmune syndrome in the subject. As used in this context, to “treat” means to ameliorate at least one symptom or clinical sign of the disorder or condition. Similarly, the term “prevent” does not require complete inhibition, but merely the delay or decrease of the onset of the disorder or condition or its symptoms. Often, myocarditis following infarction or cardiac infection results in impaired cardiac pumping function or arrhythmias, thus, a treatment can result in a reduction in cardiac inflammation and a return or approach to normal cardiac function or rhythm. As a preventative, the amount of the peptide can be sufficient to achieve any reduction in cardiac inflammation following the MI or myocardial injury.

In some embodiments, suppressing the immune response to alpha myosin HC comprises vaccinating the subject against a peptide(s) unique to alpha myosin HC. Preferably, vaccination may be done by any means of administration (for example intramuscular injection, nasal administration, oral administration or the like) of an amount of alpha myosin HC-specific peptide(s) sufficient to induce tolerance against alpha myosin HC in the subject. Standard techniques for inducing tolerance by vaccination may be used, and are to be contrasted with techniques that have the opposite effect of inducing or aggravating cardiac autoimmunity by administering the same peptide or peptides.

In some embodiments, suppressing the immune response to alpha myosin HC comprises vaccinating the subject against an immunodominant T cell epitope(s) within alpha myosin-specific peptide(s). In some embodiments, the peptide includes at least one of the unique regions in FIG. 1, and comprises an immunodominant T cell epitope.

In some embodiments, vaccination of high-risk subjects may be done prior to initiation or completion of testing of the subject for autoantibodies and/or T cell responses against to alpha myosin HC. In some embodiments, testing will be completed and a positive result for alpha myosin HC autoantibodies and/or T-cell responses against alpha myosin HC will be obtained prior to initiating suppression of the immune response to alpha myosin HC. In some embodiments, testing of the subject for autoantibodies and/or T cell responses against alpha myosin HC is not required. In some embodiments, subjects with T1D and MI or ischemic heart disease can be vaccinated.

In the case of post-myocardial injury autoimmunity caused by infection with cardiotropic pathogens, vaccination of subjects may have the most therapeutic value in areas where the disease is endemic. For example, vaccination against alpha myosin HC may be therapeutically most valuable in areas where Chagas is endemic, such as Latin America. In some embodiments, subjects at risk for developing Chagas' cardiomyopathy may be vaccinated by administering to the subject a compound to suppress an immune response to alpha myosin HC. In some embodiments, the compound administered comprises a vaccine comprising a peptide unique to alpha myosin HC in comparison to beta myosin HC. In some embodiments, subjects at risk of developing Chagas' cardiomyopathy are identified by identifying a subject who is, or is going to be, exposed to an environment in which Chagas disease is endemic.

In some embodiments, suppressing the immune response to alpha myosin HC is achieved by inducing alpha myosin HC-specific T regulatory cells. In some embodiments, the alpha myosin HC-specific T regulatory cells induced are alpha myosin HC-specific forkhead box P3-positive (FOXP3+) T regulatory cells (von Boehmer and Daniel, Nature Reviews Drug Discovery, 2013). T regulatory cells can suppress inflammatory T-cell mediated immune responses through direct contact-dependent inhibition of antigen-presenting cells and effector T cells or by the release of anti-inflammatory cytokines such as IL-10 or transforming growth factor-β (TGFβ) or by other approaches.

In some embodiments, suppressing the immune response to alpha myosin HC is achieved by a small molecule inhibitor. In some embodiments, the small molecule inhibitor can be specific to DQ8, DQ2, or other human HLA genotypes, and are capable of occupying specific structural pockets (p1, p4, p6, and p9) along the peptide binding groove on particular human MHC Class II molecules, and/or the binding groove on the equivalent mouse molecule, such as I-Ag7. In some embodiments, the small molecule inhibitor is tetraazatricyclododecane (TATD). In some embodiments, the small molecule inhibitor is an inhibitor of antigen presenting cells.

In some embodiments, any of the above methods can be applied to subjects who are not autoimmune-prone. In some embodiments, any of the above methods can be applied to disorders that do not involve T1D and/or MI.

In some embodiments, a method for treating post-myocardial injury autoimmunity is provided, the method comprises identifying a subject who has been infected with a cardiotropic pathogen, and suppressing an immune response to the alpha isoform of myosin heavy chain in the subject. In some embodiments, the method comprises identifying a subject who is at risk of being infected with a cardiotropic pathogen; and suppressing an immune response to the alpha isoform of myosin heavy chain in the subject, before they are infected (for example, a preventative treatment) and/or confirmed to have been infected with any cardiotropic pathogen.

In some embodiments, the various options noted above can be applied for these methods as well, for example, in some embodiments, suppressing the immune response to alpha myosin is not an immunosuppressive therapy. In some embodiments, the method further comprises testing the subject for autoantibodies against alpha myosin heavy chain, T-cell responses against alpha myosin heavy chain, or both. In some embodiments, suppressing the immune response to alpha myosin heavy chain is achieved by antigen-specific tolerogenic immunointervention. In some embodiments, suppressing the immune response to alpha myosin heavy chain is achieved by HLA class II-specific immunointervention. In some embodiments, suppressing the immune response to alpha myosin heavy chain is achieved by administering an alpha myosin-specific peptide(s) to the subject. In some embodiments, the subject has evidence of inflammatory cardiomyopathy due to at least one of Chagas' cardiomyopathy, post-viral myocarditis, or other cardiotrophic pathogens.

In some embodiments, the subject has been infected by a cardiotropic pathogen (for example, Coxsackievirus B3 (CB3), Hepatitis C (Matsumori, Circulation Research, 2005) or Trypanosoma cruzi (T. cruzi)).

In some embodiments, a method of treating post-myocardial injury autoimmunity in a subject is provided, the method can comprise identifying a subject who has been infected by a cardiotropic pathogen (for example, CB3 or T. cruzi), testing the subject for autoantibodies and/or T-cell responses against alpha myosin HC, and suppressing an immune response to alpha myosin HC in the subject.

Testing for Autoantibodies and/or T Cell Responses to Alpha-Myosin HC

Methods known in the art or described herein can be used test the subject for autoantibodies and/or T cell responses against α-myosin HC. These same assays can also be used as biomarkers of the therapeutic effects of antigen-specific therapies. For example, serum samples from subjects can be contacted with alpha myosin HC antigen for a sufficient amount of time and under conditions that allow binding of the alpha myosin HC antigen to any autoantibodies in the serum samples. Binding between the alpha myosin HC antigen and the autoantibodies is then detected. In some cases, autoantibodies against alpha myosin HC may also be quantified, although this is not strictly necessary.

For example, enzyme-linked immunosorbent assay (ELISA) can be used to detect the presence or absence of autoantibodies in the serum of subjects. ELISA can detect autoantibodies that bind to antigens or epitopes within the peptides immobilized on solid support (for example, a multi-well plate) by using enzyme-linked secondary antibodies, such as goat anti-human Ig Abs, and enzyme substrates that change color in the presence of enzyme-labeled antibodies.

In some embodiments, fluid-phase assays such as radioimmunoassays (RIA) can also be used to detect the presence or absence of autoantibodies α-myosin HC. For example, the gene for the alpha myosin HC antigen (or a fragment thereof) can be cloned into an expression vector, and in vitro translation can be carried out with [³⁵S]methionine to produce radiolabeled antigens. Antibody-bound radiolabeled antigens can be separated from free radiolabeled antigens with, for example, protein A-Sepharose or protein G-Sepharose beads, which bind to the antibodies. The presence of antibodies in a sample can be detected using methods known in the art and described herein.

Alternatively, the subject may be tested for T-cell responses against alpha myosin HC by ELISPOT as has been described (Lv et al, The Journal of Clinical Investigation, 2011). The subject may also be tested both for autoantibodies and T-cell responses against α-myosin HC or to immunodominant T-cell epitopes within alpha myosin HC. Such testing may be initiated or completed either before or after suppressing or beginning to suppress the immune response to alpha myosin HC. In some embodiments, the subject need not be tested for autoantibodies and/or T cell responses to alpha myosin HC, for example, if the subject is known to have T1D and/or has just recently had a MI.

### Subject Populations

Type 1 diabetes is an autoimmune disorder caused by T lymphocyte-mediated destruction of the pancreatic β cells (“insulitis”). The P-cell specificity of this autoimmune attack has been attributed to specific alleles of major histocompatibility complex (MHC) class II, most notably HLA-DQ8 (DQ8) (Stadinski, Kappler and Eisenbarth, Immunity, 2010).

Myocardial infarction is known to induce a profound inflammatory response with the influx of monocytes/macrophages and production of proinflammatory cytokines that are crucial for cardiac repair and resolve with tissue healing. While these innate immune responses are critical for cardiac repair, these same cytokines and signals from necrotic cells are known to be potent maturation factors for dendritic cells, transforming them into highly immunogenic antigen-presenting cells capable of activating adaptive immune responses.

Autoimmune-prone subjects-such as individuals with T1D or other autoimmune diseases such as celiac disease, auto-immune thyroid disease, and/or thyroiditis—can have alterations in adaptive immunity that could contribute to the development of post-infarct autoimmunity.

Data described in PCT Publication No. WO 2011/156740 A2 and herein reveal that the alpha isoform of cardiac myosin heavy chain is an autoantigen for PIA syndrome and demonstrate that induction of tolerance to alpha myosin HC prevents this disease process from occurring in humanized mice. Data described in PCT Publication No. WO 2011/156740 A2 and herein also shows that experimental induction of myocardial infarction (MI) by coronary artery ligation in 7-8 week old normoglycemic non-obese diabetic (NOD) mice, but not in age-matched control C57BL/6 mice, triggered rapid (within 1 week) development of a PIA syndrome characterized by: 1) sustained production of high-titer IgG autoantibodies targeted against cardiac myosin; 2) destructive lymphocytic infiltrates in the myocardium, similar in composition to pancreatic insulitis lesions; and 3) poor infarct healing. Data also demonstrates that myocardial ischemic injury induces cardiac autoimmunity in human patients with T ID.

The methods described herein can be used to treat post-infarction autoimmune syndrome or inflammatory cardiomyopathy caused by infectious agents or other forms of cardiac injury in an identified subject. In some embodiments, an autoimmune-prone subject who has suffered a myocardial infarction may be identified. In some embodiments, a subject who has been infected with a cardiotropic pathogen may be identified. In each case, identification of the initial class of subject can be done by standard diagnostic methods.

As used herein, an autoimmune-prone subject is a subject who has been diagnosed with an autoimmune disorder or determined to have an elevated risk of developing such a disorder. As used herein, an autoimmune disorder is a condition that occurs when the immune system mistakenly attacks and destroys healthy body tissue. Examples of autoimmune (or autoimmune-related) disorders include, but are not limited to type 1 diabetes mellitus, thyroiditis (for example, Hashimoto's thyroiditis), pernicious anemia, Addison's disease I, rheumatoid arthritis, systemic lupus erythematosus (SLE), dermatomyositis, Sjogren syndrome, lupus erythematosus, multiple sclerosis, myasthenia gravis, reactive arthritis, Grave's disease, celiac disease, Crohn's disease, acute disseminated encephalomyelitis, ankylosing spondylitis, antiphospholipid antibody syndrome, aplastic anemia, autoimmune hepatitis, autoimmune oophoritis, celiac disease, Goodpasture's syndrome, Guillain-Barre syndrome, idiopathic thrombocytopenic purpura, Kawasaki's disease, opsoclonus myoclonus syndrome, optic neuritis, pemphigus, polyarthritis, primary biliary cirrhosis, psoriasis, Reiter's syndrome, small vessel vasculitis, Takayasu's arteritis, temporal arteritis, ulcerative colitis, warm autoimmune hemolytic anemia, or Wegener's granulomatosis and idiopathic membranous nephropathy.

In some embodiments, the subject is an autoimmune-prone subject who has (for example, has been diagnosed with) an autoimmune disorder selected from the group of at least type 1 diabetes, celiac disease, and thyroiditis. Diagnosis of an autoimmune disorder or of an elevated risk of developed such a disorder can be made by a clinician using methods known in the art.

In some embodiments, the methods described herein are suitable for treating subjects who have suffered a MI shortly (for example, one week to a few months) or some time (for example, up to 12 years) prior to the application of the methods described herein. Thus, the methods can be applied any time after a subject has suffered an MI. In some cases, it can be beneficial to test the subject for autoantibodies and/or T cell responses against alpha myosin HC no earlier than 1 month after an MI to avoid false positives (for example, transient autoimmunity).

In some embodiments, the methods can be used to treat post-myocardial autoimmunity or inflammatory cardiomyopathy associated with a heart transplant. Receipt of the heart transplant can be at any time relative to identification of the subject and testing of the subject for autoantibodies and/or T cell responses to α-myosin HC. In some embodiments, testing of the subject for autoantibodies and/or T cell responses to alpha myosin HC is not required.

Diagnosis of infection by a cardiac tropic pathogen can be made by a clinician using methods known in the art. Infection by a cardiotropic pathogen need not be live or recent to identify a subject as having been infected at one time. Moreover, diagnosis of a heart condition known to be caused by infection by a cardiotropic pathogen (such as Chagas' cardiomyopathy or postviral myocarditis) may be used to determine that a subject has been infected with such a cardiotropic virus or pathogen, with no further confirmation of infection with such a pathogen.

In some embodiments, subjects with other conditions, such as postviral myocarditis or Chagas' disease, which are linked to infection by a cardiotropic pathogen (CB3 or T. cruzi, respectively) can also be treated according to the methods described herein. For example, Chagas' cardiomyopathy is thought to be a form of chronic myocarditis. Cardiac-myosin-specific CD4+ T cells have been identified in the myocardium of affected patients and are believed to play a central role in disease pathogenesis. In addition, general immunosuppression may be contradicted in patients infected with a cardiotropic pathogen, making suppression of an immune response to alpha myosin HC particularly useful in this setting.

### EXAMPLES

Below are non-limiting examples of various embodiments provided herein.

### Example 1

### Establishment of a Mouse Model of Post-Infarction Autoimmunity

Described in this example is the establishment of an experimental model that can permit detailed mechanistic studies on how autoimmune reactions contribute to cardiovascular disease complications in autoimmune-prone subjects such as individuals with type 1 diabetes or other autoimmune disorders, such as celiac disease or thyroiditis. This mouse model can also be used to confirm suppression of an immune response (including, for example, ‘antigen-specific tolerance’) against alpha-mysoin HC following a myocardial infarction and/or to test the efficacy of a specific treatment for post-infarction autoimmunity.

MI was experimentally induced by occluding the left anterior descending coronary artery in normoglycemic 7-8 wk-old NOD and control C57BL/6 (B6) mice, and the mice were followed for up to 8 to 12 weeks. To optimize survival, infarcts were induced that involved on average 20-30% of the left ventricle and were not extensive enough to result in cardiac failure in control mice.

The mice were evaluated for the presence of autoantibodies to cardiac antigens as previously described (Taylor et al, J. Immunol. 2004 Feb. 15; 172(4):2651-8; Lv et al, J. Clin Invest. 2011: 121(4): 1561-1573). None of the unmanipulated control NOD mice tested positive for cardiac autoantibodies at baseline or during the course of the study. However, starting as early as 1 week after infarction, NOD mice—but not B6 mice—developed high-titer circulating IgG autoantibodies which, by indirect immunofluorescence and confocal microscopy on healthy heart tissue, localized to the striations within myocytes, producing a distinctive myofibrillar pattern, similar to that observed in serum from HLA-DQ8 transgenic mice with spontaneous autoimmune myocarditis (Taylor et al, 2004). Notably, histological analyses of the NOD hearts 21 d after infarction showed dense lymphocytic infiltrates that were most pronounced in the peri-infarct border zone (FIG. 2A, upper left panel). In addition, direct immunofluorescence analysis revealed diffuse deposition of IgG antibodies over the entire heart, including regions remote from the infarct zone in the post-infarcted NOD hearts, whereas the post-infarcted B6 hearts were devoid of IgG deposition.

Moreover, post-MI NOD mice developed lymphocytic infiltrates in the heart, with a cellular composition (CD4+ and CD8+ T cells, and B220+ B cells; see FIG. 2A) that closely mirrored insulitis lesions in the pancreas (FIG. 2B). In addition, longer follow-up of post-MI NOD showed that PIA was associated with impaired infarct healing. For example, the infarct in post-MI NOD appeared markedly edematous with poor scar formation (FIG. 2A, upper left panel, arrow), suggesting impaired infarct healing. Over time, the histopathological changes in post-MI NOD mice also became more pronounced: by 8 wk post-MI, the infiltrates extended into the non-infarcted myocardium with further swelling and expansion of the infarct zone (FIG. 2B). By contrast, the control post-infarcted B6 hearts had no lymphocytic infiltrates and showed normal infarct healing and scar formation (FIG. 2A, lower left panel; FIG. 2B).

In this model, the severity of post-infarction autoimmunity (PIA) correlated with the size of the initial cardiac injury. The severity of PIA also correlated with the specific location of the occluding suture, with the “high” suture location (near the atrioventricular junction, at the edge of the atrial appendage) inducing the most robust PIA phenotype, but still enabling a −50% survival.

In contrast. NOD mice did not develop antibodies or myositis in response to acute necrotic skeletal muscle injuries known to stimulate muscle regeneration: cold injury (dry ice), mechanical injury (crush injury) and chemically-induced injury (cardiotoxin). These results demonstrate that the induction of PIA was specific to ischemic heart injury and was not part of a generalized response to tissue injury.

Experimental MI was also performed in humanized DQ8+NOD mice, which spontaneously develop myocarditis and cardiac autoimmunity. It was found post-MI autoimmunity was markedly augmented in DQ8+NOD mice.

### Example 2

### Sustained Production of Circulating Autoantibodies to Alpha Myosin HC

At baseline (pre-infarction), none of the NOD mice tested positive for cardiac autoantibodies. However, as early as 2 wk after MI, NOD—but not negative control B6—mice developed high titers of circulating IgG antibodies, which when tested by indirect immunofluorescence and confocal microscopy, produced a distinct myofibrillar pattern on normal heart tissue sections similar to that produced by serum from DQ8⁺NOD mice with myocarditis (FIG. 3A).

Although acute necrotic injury from MI might have been expected to result in the exposure and loss of tolerance to multiple cardiac autoantigens, it was found that post-MI NOD mice developed autoantibodies to predominantly two proteins: myosin heavy chain (myosin HC) and a second −100 kDa protein. This protein was only detectable in SDS (Laemmli) lysates and was not present in the myofibrillar heart extracts (“MFE”) that were previously used for Western blot analysis (FIG. 3B). The −100 kDa protein was expressed at high levels in cardiac muscle and skeletal muscle but was absent in lung, liver, kidney and brain. (FIG. 3C). Immunoprecipitation of this protein with serum from post-MI NOD mice, followed by excision and enzymatic digestion of the band from a gel, and analysis by tandem mass spectrometry revealed that its peptide sequences were identical to the 100 kDa structural/cytoskeletal protein, α-actinin-2 (hereafter, Actn2, FIG. 3D), which has a MW of 103,854 kDa. Actn2 is the main component of sarcomeric Z-bands where it functions to anchor actin-containing thin filaments together. Although the primary function of Actn2 is in actin binding, over 20 other different binding partners have been discovered so far, including inducible NO synthetases, PI-3 kinases, BI integrins and cardiac ion channels.

It was confirmed that Actn2 was the 100 kDa antigen recognized by post-MI NOD autoantibodies by expressing and purifying recombinant mouse Actn2 followed by Western blot confirmation. Actn2 cDNA was cloned from mouse heart mRNA using RT-PCR, produced recombinant Actn2 as a polyhistidine-tagged fusion protein in E. coli (FIG. 3E, left panel), and purified the Actn2 protein on a Ni²⁺ charged Sepharose affinity column, followed by a size-exclusion chromatography. Subsequent immunoblot assays demonstrated that post-MI NOD serum recognized the recombinant mouse Actn2 protein identical to the natively produced Actn2 protein in mouse heart lysates (FIG. 3E, right panel).

Analysis by standard ELISA and Western blotting techniques revealed that 89% (72/81) of post-MI NOD mice were positive for autoantibodies to myosin HC and Actn2. (FIG. 4A) In contrast, cardiac autoantibodies were absent or barely detectable in the post-infarcted control B6 mice (n=56) up to 12 wk post-MI (FIG. 4B). Interestingly, antibodies against Actn2 were detectable as early as 1 wk post-Mi (the earliest timepoint examined) but myocarditis mice did not develop Actn2 autoantibodies until very late in the disease course, once the mice had signs of severe congestive heart failure. The autoantibodies to cardiac myosin and Actn2 remained elevated for at least 8 wk after MI. Interestingly, NOD mice also developed autoantibodies to cardiac myosin and Actn2 after smaller infarctions (‘microinfarctions’), produced by placing a suture around the left coronary artery without permanent ligation. (FIG. 4C). These manipulations resulted in focal areas of myocardial fibrosis by Masson's trichrome staining (FIG. 5), rather than the widespread necrosis characteristic of permanent ligation. However, the prevalence and titers of cardiac autoantibodies after microinfarction was lower than those observed after full-scale MI, with <50% of microinfarcted NOD mice (6/15) exhibiting positive cardiac autoantibody titers at the end of the study period (FIG. 4C). These findings suggested that the severity of PIA correlated with the magnitude of the initial cardiac injury. The sham-operated NOD mice that received just open-chest thoracotomy, but no LAD coronary occlusion, did not develop autoantibodies or cardiac infiltrates. (FIGS. 4C and 6). Thus, the development of PIA was strictly due to myocardial injury and did not result from the nonspecific effects of open-chest surgical trauma or anesthesia (Entman et al, 2000).

As will be appreciated in light of the above and the following examples, the above mouse model and the observation of the autoantibodies to alpha myosin HC (Examples 1 and 2), can be used as a method for screening for agents that block the formation of these antibodies to the autoantibodies to alpha myosin HC (as outlined in some of the examples below).

### Example 3

### Proinflammatory T Helper Type 1 (Th1) Effector Responses to Alpha-Myosin HC

The development of circulating IgG autoantibodies to cardiac myosin and Actn2 and lymphocytic infiltrates in the myocardium suggested that signals from ischemic heart injury alone might induced adaptive immune responses in NOD mice—without any infectious triggers. To examine whether Th1 cells that are the main mediators of destruction in spontaneous myocarditis specifically target cardiac myosin and Actn2, interferon (IFN)-γ enzyme-linked immunospot (ELISPOT) assays were performed on splenocytes from 21 d post-MI NOD mice and B6 mice. These studies revealed that MI induced strong Th1 effector responses to cardiac myosin that were similar in magnitude to those found in DQ8+NOD mice with myocarditis (FIG. 7A). Cardiac-draining lymph nodes (FIG. 7B, upper panel) were also markedly enlarged by 8 wk post-MI and exhibited strong dose-dependent T cell responses to cardiac myosin that were substantially greater than those found in mesenteric lymph nodes (FIG. 7B, lower panel). None of post-MI B6 control mice showed enlarged cardiac lymph nodes or increased T cell responses to cardiac myosin (FIGS. 7A and 3B).

Surprisingly, and in stark contrast to the results for cardiac myosin, no increased T-cell responses to Actn2 in post-MI NOD mice were observed, regardless of whether IFN-γ ELISPOT or proliferation assays were used (FIG. 8). To understand this further, serial serum samples were collected bi-weekly from post-MI NOD and B6 mice, to measure the isotypes of autoantibodies specific to cardiac myosin and Actn2. (FIG. 7C). As shown in FIG. 3C, small increases of cardiac myosin-specific IgM antibodies were observed by 2 wk post-MI in both NOD and B6 mice, suggesting comparable T-cell independent immune responses in both strains. However, cardiac myosin-specific IgG1, IgG2b, and IgG2c autoantibodies were found only in NOD mice, with the peak titer to IgG1 occurring earlier than that to IgG2b and IgG2c (FIG. 7C, upper panel). In contrast, cardiac myosin-specific IgG3 and IgA responses were undetectable (FIG. 9). These studies suggested that both Th1 and Th2 cells contributed to the cardiac myosin-specific IgG antibody switch. Interestingly, the post-MI cardiac myosin-specific autoantibody isotype profiles nearly matched those of DQ8+NOD mice with spontaneous myocarditis (FIG. 7C).

By contrast, the Actn2-specific autoantibodies were almost exclusively IgG1, with little IgG2b and no IgG2c production (FIG. 7C, lower panel). This suggested a Th2 cell-dominated response and explained the lack of detection of Actn2-specific Th1 responses in post-MI NOD mice. Thus, experimentally induced MI triggered the prolonged breakdown of T and B cell tolerance to cardiac myosin in NOD mice, with resulting chronic lymphocytic infiltration in the myocardium and impaired infarct healing.

### Example 4

### Immune Tolerance to Alpha Myosin HC Prevents PIA and Promotes Normal Infarct Healing

We had previously shown that transgenic expression of alpha myosin HC in the thymus of DQ8+NOD mice induced tolerance to cardiac myosin and prevents myocarditis, demonstrating that α-myosin HC is an essential (‘initiating’) autoantigen in this spontaneous autoimmune disease process.

To test whether immune responses to cardiac myosin are also required for initiation of PIA, MI was induced in transgenic thymic alpha myosin HC-expressing TOM+TA+DQ8+NOD mice (n=4) and in control non-thymic myosin-expressing (TOM+TA− or TOM−TA+)DQ8+NOD mice (n=4) and followed the mice up to 20 days. As expected, none of the thymic alpha-myosin HC expressing TOM+TA+DQ8+NOD mice, but 3 of 4 control (TOM+TA− or TOM− TA+)DQ8+NOD mice tested positive for cardiac myosin autoantibodies pre-MI. Titers ranged from 1:100 to 1:1600 (FIG. 10A). Following MI, cardiac myosin autoantibody titers significantly increased in 3 of 4 control DQ8+NOD mice, with 2 mice developing titers to 1:6400 by 20 d post-MI (FIG. 10B). Western blotting using serum from control mice confirmed these findings, while also revealing the presence of cardiac troponin T autoantibodies (cTnT, FIG. 10B) and weaker reactivity to cardiac troponin I (cTnI). In contrast, none of the thymic myosin-expressing TOM+TA+DQ8+NOD mice developed cardiac myosin autoantibody titers when measured by ELISA, but one showed faint reactivity to cTnT by Western blotting (FIG. 10B). The two highest-titer DQ8+NOD mice demonstrated dilated hearts and massive enlargement of cardiac draining lymph nodes 20 d post-MI, other mice had normal heart sizes (FIG. 10C). Significant pathology in the control TOM−TA+DQ8+NOD mouse hearts was shown by histology, with dense lymphocytic infiltrates throughout the infarct zone, infarct expansion and lymphocytic invasion into non-infarcted myocardium (FIG. 10C, right panel). This indicated aggravated PIA in DQ8+NOD mice compared with WT NOD mice (FIG. 2, upper panel), consistent with previous reports of impaired infarct healing in mice with pre-existing myocarditis.

In contrast, none of the post-MI hearts from the alpha myosin HC-tolerant TOM+TA+DQ8+NOD mice exhibited lymphocytic infiltrates or impaired infarct healing, but instead showed normal dense scar formation (FIG. 10C, left panel). Accordingly, these findings indicate that the autoimmune response in PIA was antigen-specific and was specifically driven by the loss of tolerance to a single autoantigen, the alpha myosin HC.

### Example 5

### Discovery of a PIA Syndrome in Humans with Type 1 Diabetes

The development of a cardiac autoimmunity syndrome in post-infarcted NOD mice and its exacerbation in DQ8+NOD mice raised the possibility that some human subjects with T1D might also develop cardiac autoimmunity following MI. Since cardiac autoantibodies developed very soon after infarction in the NOD mouse model (1 week, the earliest time-point examined) and remained persistently elevated (FIG. 7C) in post-MI NOD mice, this suggested that the timing of sample collection relative to the date of the MI might not be critical.

Although the presence of autoantibodies to cardiac myosin could be clearly distinguished between pre- and post-infarcted NOD mice using indirect immunofluorescence serum staining of heart sections (FIG. 3A), Western blot (FIG. 3B), and ELISA technique (FIG. 7C), human serum did not perform as well in these assays, with unacceptable numbers of normal control subjects showing false-positivity. (FIG. 11).

### Human Cardiac Autoantigen Radioimmunobinding Assays (RIA)

Modeling on the success of “biochemical” islet autoantibody assays that are widely used in T1D screening programs, fluid-phase radioimmunoprecipitation cardiac autoantibody assays were developed using 35S-methionine-labeled in vitro transcribed and translated complementary DNAs (cDNAs) encoding human alpha myosin HC (MYH6), a major T cell autoantigen in human myocarditis, and human beta myosin HC (MYH7), which is 93% identical to alpha myosin HC and is the predominant myosin expressed in the adult human ventricle. This was followed by precipitation with protein-A/G Sepharose beads (Stewart et al, Circulation. 2010: 122: A 17040).

To validate these assays, serum reactivities were examined from 18 consecutively recruited patients with myocarditis that was established by clinical, pathological (endomyocardial biopsy), or cardiac magnetic resonance imaging (MRI) criteria). 78 consecutively recruited healthy subjects were used as negative controls. Using these new assays, it was found that 2/78 (3%) of healthy control subjects, but 5/18 (28%) patients with myocarditis tested positive for alpha or beta myosin HC autoantibodies (Table 1).

TABLE 1
Characteristics of Cardiac Autoantibody-Positive Patients

| | | | | Timing | | | | | | | |
|---|---|---|---|---|---|---|---|---|---|---|---|
| | | | | of | | | | | | | |
| | | | | sample | α- | α-MyHC | | | | | |
| | Patient | Age | | from MI | MyHC | fragments | | | β-MyHC | | |
| Diagnosis | ID | (years) | Gender | (years) | FL | S1 | S2 | LMM | FL | Actn2 | cTnI |
| T1D⁺MI⁺ | T1D-1 | 68 | F | u | − | − | − | − | − | + | − |
| | T1D-2 | 56 | F | 7 | − | + | + | − | − | − | − |
| | T1D-5 | 39 | F | 2 | ⊕ | − | − | − | ⊕ | + | − |
| | T1D-6 | 59 | M | 4 | ⊕ | + | − | − | ⊕ | − | − |
| | T1D-7 | 67 | F | 5 | − | + | − | − | − | − | − |
| | T1D-8 | 61 | M | 7 | ⊕ | + | − | − | ⊕ | − | + |
| | T1D-9 | 69 | M | 8 | − | + | − | − | − | − | − |
| | T1D-10 | 57 | F | 5 | − | + | − | − | − | − | − |
| | T1D-12 | 52 | M | u | − | + | − | − | − | − | − |
| | T1D-13 | 19 | M | 5 | − | − | − | + | − | − | − |
| | T1D-14 | 46 | F | 8 | − | + | − | − | − | − | − |
| | T1D-15 | 66 | F | u | − | − | − | − | − | + | − |
| | T1D-16 | 59 | M | 3 | ⊕ | + | − | − | ⊕ | − | − |
| | T1D-17 | 55 | M | 3 | − | − | − | − | − | − | + |
| | T1D-18 | 54 | F | 0.5 | − | + | − | − | − | − | + |
| T2D⁺MI⁺ | T2D-4 | 63 | M | 10 | − | − | − | + | − | − | − |
| | T2D-5 | 57 | M | 0.9 | − | − | − | − | | − | − |
| | T2D-20 | 59 | F | 11 | − | + | − | − | − | − | + |
| | | | | Dura- | | | | | | | |
| | | | | tion of | | | | | | | |
| | | | | symp- | | | | | | | |
| | | | | toms | | | | | | | |
| | | | | (days) | | | | | | | |
| Myocarditis | M-1 | 36 | M | 6 | ⊕ | − | + | + | ⊕ | − | − |
| (nondiabetic) | M-2 | 51 | F | 15 | − | − | − | − | − | − | + |
| | M-3 | 50 | F | 137 | − | + | + | − | − | − | − |
| | M-4 | 21 | M | 196 | − | − | − | + | − | − | − |
| | M-5 | 33 | M | 4 | ⊕ | − | + | + | ⊕ | − | − |
| | M-6 | 26 | M | 866 | − | + | − | − | − | − | − |
| | M-7 | 21 | M | 8 | ⊕ | + | − | − | ⊕ | − | − |
| | M-8 | 19 | M | 13 | − | + | − | + | − | + | − |
| | M-9 | 20 | F | 4 | − | − | + | − | − | − | − |
| | M-10 | 55 | M | 5 | ⊕ | + | − | − | ⊕ | − | − |
| | M-11 | 37 | F | 11 | ⊕ | + | − | − | ⊕ | − | − |
| | M-12 | 20 | M | 1 | − | − | − | − | − | + | − |

Autoantibody reactivity was analyzed in a radioimmunoprecipitation assay format for full-length MYH6 (α-MyHC FL); S I, S2, and LMM fragments of α-MyHCMYH6: full-length MYH7 (β-MyHC FL); a-actinin-2 (Actn2), cardiac troponin I (cTnI). −, negative for antibody reactivity: +, positive for antibody reactivity; u, timing of MI unknown.

Although the prevalence of myosin HC autoantibodies in myocarditis patients was relatively low, the patterns of reactivity were striking with positivity to both human alpha and beta myosin HC (Table 1, circles), similar to the dual isoform reactivity of serum from DQ8+NOD mice with myocarditis but distinctive from the two healthy control subjects who had reactivity only to a single myosin HC isoform. Interestingly, the duration of symptoms was relatively short (mean=7 d; range, 4-11d) in the full-length alpha and beta myosin HC autoantibody-positive myocarditis patients (Table 1), suggesting an acute disease process.

To further refine these assays, RIAs were developed using cDNA fragments corresponding to the three major functional domains of α-myosin HC (subfragment 1 (S1), subfragment 2 (S2), and light meromyosin (LMM)) (FIG. 12). Because of the large size of alpha myosin HC, assays were established with three overlapping fragments encompassing the entire protein (S I, S2, LMM). Overlapping fragments of nucleic acid encoding the S1, S2 and LMM domains of human MYH6 (alpha myosin HC) were amplified by PCR using the primers shown in Table 2 (shown below), and cloned. The S1 polypeptide used in this example corresponds approximately to the first 865 amino acids of human MYH6 (for example, Genbank Accession No. NP_{—}002462.2). The S2 polypeptide used in this example corresponds approximately to amino acid numbers 822 to 1327 and the LMM polypeptide used in this example corresponds approximately to amino acid numbers 1237 to 1940.

TABLE 2
Primers used to generate MYH6 cDNA fragments by PCR
amplification
Table 1: Primers used to generate MYH6 cDNA
fragments by PCR amplification

| | | SEQ ID |
|---|---|---|
| Fragment | Sequence | NO: |
| S1 | F: TTGCACTCGAGAATTCCGAGATGACCGATGCCCAGATGG | 1 |
| | R: TACCACGCGTGCGGCCGCTCACAGCGTCTCTTTGATGCG | 2 |
| S2 | F: TTGCACGTCGACACCATGGCCTTCATGGGGGTCAAG-3′ | 3 |
| | R: TACCACTGCGGCCGCTCACGCCTTGCCCTCCTCCTCCAG | 4 |
| LMM | F: TTGCACGTCGACAACATGGAGCAGATCATCAAGGCC | 5 |
| | R: TACCACGCGTGCGGCCGCAGGTTCCCGAGGCAGTGTCAC | 6 |

Each cDNA or fragment was cloned into an expression vector (for example, pCMV-TnT, Promega). All plasmid clones were sequenced completely to verify that no sequence errors had been introduced and also to confirm the orientation of the clone in order to express in vitro either from T7 or SP6 promoter.

The autoantigens were in vitro translated with [35S]methionine as follows. Plasmid DNA (2 g) was incubated in a 40 μï of TnT T7 or SP6 quick coupled transcription/translation (Promega, Madison, USA) with 2 μï of [35S]-methionine (1000 Ci/mmol; 10 mci/ml; GE Healthcare, Piscataway, USA) and made up the total volume of the reaction to 50 μï with nuclease free water. The reaction was incubated for 90 minutes at 30° C. Gel electrophoresis was used to confirm that the translated product resulted in protein band of the appropriate size. Patient samples were screened in a standard radioassay format using Protein A bound to Sepharose beads in 96-well membrane filtration plates to separate autoantibody-bound from free 35S-labeled autoantigens.

With these fragment assays, the sensitivity of cardiac myosin autoantibody detection almost doubled with 10/18 (56%) of myocarditis patients testing positive for one or more fragments (Table 1), compared to 3/78 healthy controls (4%, P<0.0001). Based on these results, RIAs were developed for the detection of autoantibodies to human Actn2 and human cardiac troponin I (cTnI). Cardiac troponin I was of special interest because it has been implicated as an autoantigen in mouse models of autoimmune cardiomyopathy,) and autoantibodies to cTnI have been reported in a subset of human patients immediately post-MI.

### Post-MI Cohort and Results

Subjects in the post-MI cohort included 18 consecutively recruited post-MI with T1D, mean age 55±12 years, 56% (10/18) females with a mean time interval from MI to autoantibody testing=4.5 years (range 0.3-8 years): and, as controls, 20 consecutively recruited type 2 diabetes (T2D) post-MI patients, 70% males (14/20), mean age 60±10 years with mean time interval from MI to antibody testing=8.1 years (range 0.1-20 years) (Table 3, shown below). Diagnosis of T1D was made by clinical history. High resolution HLA-DQB genotyping confirmed diagnosis of T1D, with 16/18 subjects (89%) testing positive for high risk-T1D genotypes, DQB 1*0201 or DQB 1*0302. (Table 3).

TABLE 3
Characteristics of T1D and T2D post-MI cohorts.

| | | | Post- | HLA Alleles | | | |
|---|---|---|---|---|---|---|---|
| | Sex | Age | MI | DRB1- | DQB1- | DRB1- | DQB1- |
| ID | (M/F) | (y) | (y) | Allele 1 | Allele 1 | Allele 2 | Allele 2 |
| T1D-1 | F | 68 | u | 3 | 0201 | 4 | 0302 |
| T1D-2 | F | 56 | 7 | 1 | 0501 | 4 | 0302 |
| T1D-3 | F | 49 | 1.3 | 3 | 0201 | 3 | 0201 |
| T1D-4 | M | 60 | 8 | 4 | 0302 | 4 | 0302 |
| T1D-5 | F | 39 | 2 | 3 | 0201 | 4 | 0302 |
| T1D-6 | M | 59 | 4 | 3 | 0201 | 3 | 0201 |
| T1D-7 | F | 67 | 5 | 3 | 0201 | 3 | 0201 |
| T1D-8 | M | 61 | 6 | 3 | 0201 | 4 | 0302 |
| T1D-9 | M | 69 | 8 | 4 | 0301 | 4 | 0302 |
| T1D-10 | F | 57 | 5 | 1 | 0501 | 4 | 0302 |
| T1D-11 | F | 61 | 0.3 | 3 | 0201 | 15 | 0502 |
| T1D-12 | M | 52 | u | 3 | 0201 | 4 | 0302 |
| T1D-13 | M | 19 | 5 | 3 | 0201 | 4 | 0302 |
| T1D-14 | F | 46 | 8 | 1 | 0501 | 8 | 0501 |
| T1D-15 | F | 66 | u | 1 | 0501 | 4 | 0302 |
| T1D-16 | M | 59 | 3 | 11 | 0603 | 4 | 0302 |
| T1D-17 | M | 55 | 3 | 7 | 0202 | 14 | 0503 |
| T1D-18 | F | 54 | 0.5 | 17 | 0201 | 9 | 0303 |
| T2D-1 | M | 55 | 6 | 7 | 0202 | 13 | 0603 |
| T2D-2 | F | 68 | u | 3 | 0201 | 14 | 0503 |
| T2D-3 | M | 63 | 9 | 15 | 0602 | 15 | 0602 |
| T2D-4 | M | 63 | 10 | 7 | 0202 | 11 | 0301 |
| T2D-5 | M | 57 | 0.9 | 13 | 0603 | 11 | 0301 |
| T2D-6 | M | 45 | 11 | 3 | 0201 | 13 | 0609 |
| T2D-7 | F | 61 | 7 | 4 | 0301 | 11 | 0301 |
| T2D-8 | M | 80 | 5 | 7 | 0202 | 4 | 0302 |
| T2D-9 | M | 57 | 20 | 7 | 0202 | 13 | 0603 |
| T2D-10 | M | 60 | 15 | 1 | 0501 | 13 | 0604 |
| T2D-11 | M | 36 | 5 | 3 | 0201 | 1 | 0501 |
| T2D-12 | M | 64 | 12 | 4 | 0301 | 4 | 0301 |
| T2D-13 | M | 56 | 0.3 | 7 | 0202 | 15 | 0602 |
| T2D-14 | F | 62 | 13 | 1 | 0501 | 1 | 0501 |
| T2D-15 | M | 60 | 0.08 | 15 | 0602 | 11 | 0301 |
| T2D-16 | M | 61 | 6 | 1 | 0501 | 15 | 0602 |
| T2D-17 | F | 54 | 17 | 14 | 0503 | 4 | 0302 |
| T2D-18 | M | 59 | 4 | 7 | 0303 | 9 | 0303 |
| T2D-19 | F | 78 | 0.8 | 1 | 0501 | 15 | 0602 |
| T2D-20 | F | 59 | 11 | 1 | 0501 | 8 | 0301 |

Patient and control sera were tested for binding to [35S]-labeled cardiac autoantigens in a radioimmunoassay format. For alpha myosin HC, beta myosin HC, and cTnI, RIA was performed in 50 μl of immunoprecipitation (IP) buffer containing 20 mM Tris-HCl pH 7.4, 150 mM NaCl, 1% (v/v) Triton X-100, and 10 μg/ml aprotinin (Sigma); for Actn2, 0.06 mM CaCl₂, 1 mM MgCl₂, 1.6 mM KCl were added to the IP buffer; and for alpha myosin HC fragments, 0.5% NP-40 was used instead of Triton X-100 in IP buffer. Human serum was then added to a final dilution of either 1:5 (for alpha or beta myosin HC) or 1:25 (for alpha myosin HC fragments, cTnI, and Actn2).

RIA reactions contained 400,000 counts per minute (cpm) for full-length alpha or beta myosin HC; 50,000 cpm for both alpha myosin HC fragments and Actn2; and 20,000 cpm for cTnI.

The samples were then incubated in 1.5 ml microcentrifuge tubes at room temperature for 2 h prior to overnight incubation at 4° C. with constant shaking. 50 μl of protein A/G (50% A/8% G) Sepharose 4 Fast Flow beads (GE Healthcare) were then added to the microcentrifuge tubes and incubation continued for 1 h at 4° C. The protein A/G Sepharose-antibody complexes were collected by centrifugation, washed with IP buffer, and immunoprecipitated radioactivity was measured in a LS 6500 scintillation counter (Beckman Coulter). The RIAs for alpha myosin HC fragments, Actn2, and cTnI were carried out in a 96 well Unifilter plates (Whatman). 50 μl of protein A/G (50% A/8% G) Sepharose 4 Fast Flow beads and overnight incubated antigen-antibody complexes were added to each well and incubated for 1 h at 4° C. The protein A/G Sepharose-antibody complexes were centrifuged and washed with IP buffer at 4° C. The beads were then resuspended in scintillation fluid (Ultima Gold F, Perkin Elmer), and the immunoprecipitated radioactivity was measured in an 1450 MicroBeta scintillation counter (Perkin-Elmer).

These studies revealed that serum from 15/18 (83%) post-MI T1D patients tested positive to autoantibodies to >1 cardiac antigen, in contrast to 3/20 (15%) post-MI T2D patients and 3/78 (4%) healthy control subjects (T1D post-MI vs T2D post-Ml, p=0.0001; T1D post-MI vs healthy controls, p<0.0001; T2D post-MI vs healthy controls, p=0.1834) (Table 1).

Furthermore, the prevalence and specificities of the cardiac autoantibodies were strikingly similar between the post-MI T1D and myocarditis patients, despite significant differences in the etiologies and conditions of the two conditions. (FIG. 12B and Table 1). Importantly, there was predominant targeting of cardiac myosin in both (Table 1). In particular, it was found that 4 of 18 (22%) post-MI T1D subjects tested positive for autoantibodies to both alpha and beta myosin HC, which was also a feature of myocarditis serum, while sera from post-MI T2D patients and healthy control subjects (Table 1) tested negative for these autoantibodies.

It was also found that serum in 10 of 12 (83%) post-MI T1D patients who tested positive for cardiac myosin autoantibodies, either in the full-length or fragment assays, recognized the alpha myosin HC S1 domain (Table 1). The S1 domain was also recognized by serum samples from myocarditis patients, albeit at lower prevalence (6/10 cardiac myosin autoantibody-positive subjects). Importantly, since the serum samples from the post-MI T1D subjects were obtained many years post-MI, the cardiac autoantibodies appeared to be persistent. Indeed, in a limited longitudinal follow-up study of two post-MI T1D patients (T1D-2 and T1D-8, Table 1), cardiac myosin autoantibodies were detected approximately 1 y after the initial serum sample collection, confirming autoantibody persistence.

### Example 6

### Cardiac Magnetic Resonance Imaging (MRI) Confirmation of Myocarditis in a Post-MI T1D Patient Who Tested Positive for Cardiac Autoantibodies

We also obtained corroborating MRI evidence of myocardial inflammation in one of the T1D subjects, a 61-yr old male (T1D-8) who was positive for multiple cardiac autoantibodies, including alpha-myosin HC (Table 1; FIGS. 13A and B). The patient's cardiac history showed an inferior MI approximately 6 y prior to entering the study, with initial preserved left ventricular ejection function, followed by subsequent development of cardiac dysfunction and a progressive decline in ejection fraction not explained by the extent of his coronary artery disease. Cardiac MRI using conventional contrast agents on T2-weighted black-blood short-tau inversion recovery (STIR) technique showed diffuse myocardial inflammation. (FIG. 13A, left panel). Consistent with myocardial edema, the T2-signal intensity was notably elevated, and was similar in severity to that of positive control myocarditis patient (M-5), a 33-yr old male who presented with acute myocarditis but no significant past medical history. (Table 1; FIG. 13A, right panel).

Myocardial inflammation was also observed comparing T2* images before (FIGS. 13Bi and 13Bii, at echo times of 1.4 and 21 msec, respectively) and after (FIGS. 13B (section iii) and 13B (section iv), at echo times of 1.4 and 21 msec, respectively) injection of the iron-oxide agent, Ferumoxytol, which is avidly taken up by macrophages. T2* by cardiac MRI is a decay constant which is inversely proportional to uptake of the iron-oxide contrast media by myocardial tissue. At 24 hr after injection, there was evidence of accumulation of iron-oxide in the myocardium, indicated by the decay constants of T2* (decay curves shown in FIGS. 13B (section v) and 13B (section vi)), 24 msec from before injection vs. 9.4 msec 24 hours after injection). This was strongly indicative of increased uptake of iron-contrast by an ongoing inflammatory reaction in this patient's myocardium. This patient experienced sudden cardiac death less than 2 yr later.

### Example 7

### Development of Cell-Based Assays to Screen for Inhibitors of DOS-Restricted Cardiac Myosin Presentation to Autoreactive T Cells and Identification of a Small Molecule Inhibitor

Recent advances have identified small molecules targeting the MHC class II antigen presentation pathway—specifically the presentation insulin peptide B9-23 to pathogenic T cells in NOD mice. (Michels et al., Journal of Immunology, 2011). These molecules were discovered using an in silico molecular docking program to screen a large “druglike” chemical library to define small molecules capable of occupying specific structural pockets along the NOD I-Ag7 binding groove.

To assess the potential efficacy in the setting of post-infarct autoimmunity, we created a lacZ-inducible alpha myosin HC-specific CD4⁺ T-cell hybridoma by fusing Clone E, a pathogenic DQ8-restricted CD4⁺ T cell clone isolated from a myocarditic heart (Lv H et al., J Clin Invest, 2011) (FIGS. 14A-D), and BWZ.36 fusion partner (Karttunen J et al., Proc Natl Acad Sci USA, 1992), and successfully generated the hybrid, BWZ.36.CE. Because DQ8 provides susceptibility to human T1D possibly through presenting the insulin B:9-23 epitope to T cells, we compared alpha myosin HC presentation to that of insulin on DQ8-expressing antigen presenting cells (APCs). Tetraazatricyclododecane (TATD) is a small molecular blocker of the presentation of insulin on both I-A^{g7} and DQ8 in vitro as well as in vivo (Michels et al., Journal of Immunology, 2011). The results demonstrated that TATD was able to completely block alpha myosin HC presentation to DQ8 molecules in vitro in a dose-dependent manner (FIGS. 15A-C) and that it did so in a manner analogous to the blocking effect on insulin B9:23 presentation to DQ8.

Because TATD is efficacious in preventing T1D in NOD mice (Michels et al., Journal of Immunology, 2011) and TATD more potently blocks DQ8 than I-A^{g7} molecules, these findings indicate that TATD (and similar small molecule inhibitors) can be used to suppress an immune response against alpha myosin HC and thereby treat post-infarction autoimmune syndrome in T1D patients. Similarly, in light of the above, the results indicate that the molecule can be used for treatment of post-cardiac infection autoimmunity. The above method, while applied to confirm the effectiveness of TATD, can also be used to screen for other molecules and methods that provide the same result (suppressing an immune response against α-myosin HC).

### Example 8

### The DQ8-Restricted Myocarditis-Associated Epitope Localizes to Amino Acids Unique to the Alpha Myosin HC and Resides in the S2/LMM Domain

To begin to identify the epitope(s) of cardiac myosin responsible for post-infarction autoimmunity in T1D subjects who are DQ8⁺, a lacZ and GFP-inducible α-Myosin HC-specific CD4⁺ T-cell hybridoma was made by fusing Clone E, the pathogenic DQ8-restricted CD4⁺ T cell clone isolated from a myocarditic heart (Lv H et al., J Clin Invest, 2011) and BWZ.36 hybridoma partner with NFAT-driven expression of Green Flourescence Protein (GFP) and LacZ (Karttunen J et al., Proc Natl Acad Sci USA) to generate the hybridoma, BWZ.36.CE.GFP (FIG. 16). Myosin subfragments were purified from heart tissue using enzymatic digestion and ion-exchange chromatography (Margossian et al., Methods in Enzymology, 1982).

Using the Clone E-GFP hybridomas, responses to purified antigens containing cardiac myosin, S1 subfragment, or S2/LMM subfragments were tested and it was determined that the Clone E-GFP hybridomas responded to the cardiac myosin and S2/LMM subfragments, but not the S1 subfragment. (FIG. 17). It was further determined that the fragment of “S2-LMM” contains a small region of peptides within the junction of S1/S2 that contains some amino acids that are unique to alpha myosin-HC and which belong to S1.

As will be appreciated by one of skill in the art, the above method can be repeated with any of the 10 amino acid long peptides that contain any of the identified amino acid residues that are identified as unique to alpha-myosin over beta-myosin, as shown in FIG. 1.

### Example 9

### Treatment of a T1D Patient Who has Suffered a MI

Post-infarction autoimmune syndrome (the proposed pathogenesis of which is shown in FIG. 18) can be treated in a type 1 diabetic. One first identifies the patient as having T1D and as having suffered a myocardial infarction. One then tests the patient for autoantibodies against the alpha myosin heavy chain using the RIA specific to alpha myosin HC described herein. Alternatively, the subject may be tested for T-cell responses against alpha myosin HC, or tested for both autoantibodies and T-cell responses against alpha myosin HC.

If the patient displays such autoantibodies, one can then suppress the patient's immune responses to alpha myosin HC. Suppression of the immune response to alpha myosin HC can be achieved by vaccinating the patient against a peptide unique to alpha myosin HC (as shown in FIG. 1). Alternatively, suppression of the immune response to alpha myosin HC can be achieved by using a small molecule inhibitor, such as TATD.

### Example 10

### Treatment of a Subject with Celiac Disease Who has Suffered a MI

A post-infarction autoimmune syndrome can be treated in a patient with celiac disease.

One first identifies the patient as having celiac disease and as having suffered a myocardial infarction. One then tests the patient for autoantibodies against the alpha myosin HC using the RIA specific to alpha myosin HC described herein. Alternatively, the subject may be tested for T-cell responses against alpha myosin HC, or tested for both autoantibodies and T-cell responses against alpha myosin HC.

If the patient displays such autoantibodies, one can then suppress the patient's immune responses to alpha myosin HC. Suppression of the immune response to alpha myosin HC can be achieved by vaccinating the patient against a peptide(s) (or, in the alternative, mimetopes) unique to alpha myosin HC (as shown in FIG. 1). Alternatively, suppression of the immune response to alpha myosin HC can be achieved by using a small molecule inhibitor, such as TATD.

### Example 11

### Treatment of a Subject with Thyroiditis Who has Suffered a MI

A post-infarction autoimmune syndrome can be treated in a patient with thyroiditis.

One first identifies the patient as having thyroiditis and as having suffered a myocardial infarction. One then tests the patient for autoantibodies against the alpha myosin HC using the RIA specific to alpha myosin HC described herein. Alternatively, the subject may be tested for T-cell responses against alpha myosin HC, or tested for both autoantibodies and T-cell responses against alpha myosin HC.

If the patient displays such autoantibodies, one can then suppress the patient's immune responses to alpha myosin HC. Suppression of the immune response to alpha myosin HC can be achieved by vaccinating the patient against a peptide(s) unique to alpha myosin HC (as shown in FIG. 1). Alternatively, suppression of the immune response to alpha myosin HC can be achieved by using a small molecule inhibitor, such as TATD.

### Example 12

### Treatment of a Subject Infected with a Cardiotropic Pathogen

Post-myocardial injury autoimmunity can be treated in a patient who has been infected by a cardiotropic pathogen (for example, CB3, Hepatitis C, HIV or T. cruzi).

One first identifies the patient as having been infected with a cardiotropic pathogen. One then tests the patient for autoantibodies against the alpha myosin heavy chain using the RIA specific to alpha myosin HC described herein. Alternatively, the subject may be tested for T-cell responses against alpha myosin HC, or tested for both autoantibodies and T-cell responses against alpha myosin HC.

If the patient displays such autoantibodies, one can then suppress the patient's immune responses to alpha myosin HC. Suppression of the immune response to alpha myosin HC can be achieved by vaccinating the patient against a peptide unique to alpha myosin HC (as shown in FIG. 1). Alternatively, suppression of the immune response to alpha myosin HC can be achieved by using a small molecule inhibitor, such as TATD.

### Example 13

### Administration of an Alpha Myosin HC-Specific Peptide to a Subject

The present example is directed to the suppression of the immune response to alpha myosin HC.

An alpha myosin HC-specific peptide is administered to a subject. The alpha myosin HC-specific peptide(s) comprises amino acids unique to the alpha isoform (i.e. that are not found in the beta isoform). The identified peptide sequence is at least 10 amino acids long and is selected from the options in FIG. 1. The amount of the peptide administered is sufficient to suppress an immune response against alpha myosin HC that comprises that peptide sequence.

Any method of administering the alpha myosin HC-specific peptide(s) may be used, for example, administration may be done by intramuscular injection, by nasal administration, by oral administration, or the like.

In some embodiments, the peptide(s) are immunodominant T-cell epitopes.

### Example 14

### Method of Screening for Alpha Myosin HC-Specific Peptide(s)

As indicated above, the various mouse models and methods can be used to confirm which of the residues that are different between alpha and beta myosin, shown in FIG. 1, are relevant for the development of post-myocardial injury autoimmunity and/or the PIA response.

Once an initial peptide fragment is identified as having a distinct residue adequate for these purposes, variations of the peptide (in particular, a series of progressive, overlapping peptides, all containing the residue identified as different between alpha myosin and beta myosin) can be rescreened to determine which particular peptide (and the epitope in the peptide) is the most immunogenic.

A sample assay for determining which of the residues are most relevant for the development of post-myocardial injury autoimmunity and/or the PIA response (that is, epitopes recognized by the majority of cardiac-myosin specific T cells in the peripheral blood of patients) can be found in Lv, Journal of Clinical Investigation, 2011. As described in Lv, Journal of Clinical Investigation, 2011, T cell autoreactivity against or tolerance to alpha myosin HC can be assessed by ex vivo IFN-γ and IL-10 ELISPOT assays on fresh peripheral blood mononuclear cells. As described therein, fresh human PBMCs were isolated on density gradients from anti-coagulated human whole blood samples. PBMCs were adjusted to 2×10⁶/ml in 15 ml polypropylene tubes in 0.5 ml of complete HL-1 medium supplemented with 10% human AB serum (Gemini Biotech) and stimulated for 42 h with the indicated antigens. 96-well plates (Nunc Maxisorp) were pre-coated with 4 μg/ml anti-human IFN-γ (Endogen) or IL-10 in PBS overnight at 4° C., washed with PBS and blocked for 1 hour at 37° C. with PBS/1% BSA. Each sample was washed and resuspended in 0.6 ml complete HL-1 medium and ˜300,000 cells/100 μl were dispensed in triplicate into wells of the ELISPOT plates. After 16 h incubation, ELISPOT plates were washed and 2 μg/ml biotinylated anti-human IFN-γ antibody was added to each well and incubated for 2 hours at 37° C. Plates were washed and alkaline phosphatase-conjugated streptavidin was added to each well and incubated for 2 hours at 37° C. The plates were washed and NBT solution was added to each well for 45 seconds at room temperature. The reactions were stopped by washing the plates under tap water. Spots were counted using an automated immunospot reader (Cellular Technology Ltd).

### Example 15

### Vaccination of T1D Patient at Time of MI

A subject at risk of post-infarction autoimmune syndrome can be vaccinated against the disorder.

One first identifies the subject as having T1D and as having a myocardial infarct based on standard criteria. One then administers to the subject an adequate amount of one or more of the peptides that include at least one of the residues noted in FIG. 1, to induce tolerance to the alpha myosin heavy chain. The impact of any PIA, following MI, will be reduced due to the vaccination.

### Example 16

### Vaccination of Subjects at Risk of Developing Chagas' Cardiomyopathy in Endemic Areas

A subject at risk for developing Chagas' cardiomyopathy can be vaccinated against the disorder.

One first identifies the subject as being at risk for developing Chagas' cardiomyopathy because the subject is or is going to be exposed to an environment in which Chagas disease is endemic. One then administers to the subject an adequate amount of one or more of the peptides that include at least one of the residues noted in FIG. 1, to vaccinate the subject against the alpha myosin heavy chain. The impact of any Chagas' cardiomyopathy or likelihood of developing Chagas' cardiomyopathy, in subjects who are later infected, will be reduced due to the vaccination.

### Example 17

### Treatment of Subject with Giant-Cell Myocarditis Receiving a Heart Transplant

It has been reported that ˜20% of patients with giant-cell myocarditis have co-existent autoimmune disorders and furthermore giant cell myocarditis is known to recur in transplanted hearts (Cooper, New England Journal of Medicine, 2009).

A subject who has received or will receive a heart transplant is treated by suppressing the immune response against alpha myosin HC. One first identifies the subject with giant-cell myocarditis as having received or going to receive a heart transplant. One then suppresses the subject's immune responses to alpha myosin HC. Optionally, one can test the subject for autoantibodies against alpha myosin HC and/or T cells responses against alpha myosin HC before suppressing the immune response against alpha myosin HC. If the subject displays such autoantibodies, and/or tests positive for such T cell responses, one can then suppress the patient's immune responses to alpha myosin HC. Alternatively, testing of the subject for autoantibodies against alpha myosin heavy chain and/or T cells responses against alpha myosin HC is not required.

Suppression of the immune response to alpha myosin HC can be achieved by vaccinating the patient against a peptide unique to alpha myosin HC (as shown in FIG. 1). Alternatively, suppression of the immune response to alpha myosin HC can be achieved by using a small molecule inhibitor to HLA class II molecules, such as TATD.

While particular forms of the invention have been described, it will be apparent that the invention can be embodied in other specific forms without departing from the spirit and scope thereof.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments.

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls. So there can be no question, Gottumukkala, et al., Science Translational Medicine, 2012, “Myocardial Infarction Triggers Chronic Cardiac Autoimmunity in Type 1 Diabetes” is hereby incorporated by reference in its entirety, including all figures and supplementary materials for the reference.

### EQUIVALENTS

The foregoing description and Examples detail certain embodiments. It will be appreciated, however, that no matter how detailed the foregoing may appear in text, the invention may be practiced in many ways and the invention should be construed in accordance with the appended claims and any equivalents thereof.

What is claimed is:

1. A method for treating post-myocardial injury autoimmunity in a subject, the method comprising:
identifying an autoimmune-prone subject who has suffered a myocardial injury; and
suppressing an immune response to the alpha isoform of myosin heavy chain (“alpha-myosin”) in the subject.

2. The method of claim 1, further comprising testing the subject for autoantibodies and/or T-cell responses against alpha myosin.

3. The method of claim 1, wherein the autoimmune-prone subject has a disorder selected from the group consisting of at least one of: type I diabetes, celiac disease, thyroiditis, and autoimmune thyroid disease.

4. The method in claim 1 wherein the myocardial injury comprises a myocardial infarction.

5. The method in claim 1 wherein the autoimmune-prone subject has ischemic heart disease.

6. The method in claim 1 wherein the autoimmune-prone subject has received a heart transplant.

7. The method in claim 1 wherein the autoimmune-prone subject has inflammatory cardiomyopathy.

8. The method in claim 1 wherein the autoimmune-prone subject has been infected or is at risk of being infected with a cardiotropic pathogen.

9. The method of claim 1, wherein suppressing the immune response to alpha myosin is not an immunosuppressive therapy.

10. The method of claim 1, wherein suppressing the immune response to alpha myosin is achieved by antigen-specific tolerogenic therapy.

11. The method of claim 1, wherein suppressing the immune response to alpha myosin is achieved by HLA class II-specific immunointervention.

12. The method of claim 1, wherein suppressing the immune response to alpha myosin is achieved by administering an alpha myosin-specific peptide to the subject.

13. The method of claim 1, wherein suppressing the immune response to alpha myosin comprises vaccinating the subject against a peptide unique to alpha myosin.

14. The method of claim 1, wherein suppressing the immune response to alpha myosin is achieved by inducing alpha myosin-specific T regulatory cells.

15. The method of claim 1, wherein suppressing the immune response to alpha myosin is achieved by a small molecule inhibitor to a HLA class II peptide binding pocket.

16. The method of claim 1 wherein the subject is a human.

17. A method for treating post-myocardial injury autoimmunity, the method comprising:
identifying a subject who has been infected or is at risk of being infected with a cardiotropic pathogen; and
suppressing an immune response to the alpha isoform of myosin heavy chain (“alpha-myosin”) in the subject.

18. The method in claim 17 wherein the subject has evidence of inflammatory cardiomyopathy due to at least one of Chagas' cardiomyopathy, post-viral myocarditis, or other cardiotrophic pathogens.

19. The method of claim 17, wherein suppressing the immune response to alpha myosin is achieved by administering an alpha myosin-specific peptide to the subject.

20. An alpha myosin-specific peptide comprising a contiguous stretch of at least 8 amino acids of SEQ ID NO: 1 and wherein the alpha myosin-specific peptide includes one or more of the following amino acids: 2, 4, 5, 8, 14, 33, 34, 35, 36, 37, 44, 52, 60, 65, 77, 103, 107, 110, 111, 135, 136, 197, 205, 209, 210, 211, 212, 213, 283, 304, 314, 319, 320, 327, 335, 348, 349, 350, 360, 367, 381, 417, 422, 424, 425, 435, 554, 562, 574, 586, 592, 596, 608, 618, 619, 623, 624, 627, 630, 631, 632, 633, 634, 636, 639, 640, 666, 680, 682, 730, 744, 790, 794, 797, 799, 801, 803, 806, 807, 811, 847, 853, 859, 861, 864, 901, 954, 1014, 1021, 1023, 1025, 1076, 1079, 1081, 1086, 1088, 1089, 1091, 1092, 1094, 1095, 1101, 1103, 1104, 1113, 1250, 1251, 1258, 1261, 1263, 1265, 1268, 1272, 1275, 1277, 1290, 1294, 1309, 1314, 1325, 1401, 1520, 1521, 1524, 1525, 1537, 1540, 1593, 1613, 1733, 1734, 1737, 1741, 1826, 1860, 1931, 1933, 1934, 1935, 1936, 1937, 1938, and 1939 of SEQ ID NO: 1, and wherein the peptide comprises a T cell immunodominant epitope.


SEQUENCE LISTING (1 sequence)

SEQ ID NO: 1 - protein, 1939 aa, Homo sapiens
MTDAQMADFGAAAQYLRKSEKERLEAQTRPFDIRTECFVPDDKEEFVKAKILSREGGKVIAETENGKTVTVKEDQVLQQNPPKFDKIEDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQVLALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMESDLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE